(19) United States
(12) Patent Application Publication
(10) Pub. No.: US 2003/0139431 A1
(43) Pub. Date: Jul. 24, 2003

(54) Guanidines which are agonist/antagonist ligands for neuropeptide FF (NPFF) receptors

(76) Inventors: Joel K. Kawakami, Newfoundland, NJ (US); John Wetzel, Fairlawn, NJ (US); Lakmal W. Boteju, Cedar Grove, NJ (US); Michael J. Konkel, Garfield, NJ (US); Honghe Wan, Kearny, NJ (US); Stewart A. Noble, Lake Forest, IL (US)

Correspondence Address: Cooper & Dunham LLP, 1185 Avenue of the Americas, New York, NY, 10036, US

(21) Appl. No.: 10/253,946

(22) Filed: Sep. 21, 2002

Related U.S. Application Data
(60) Provisional application No. 60/324,559, filed on Sep. 24, 2001.

(51) Int. Cl. A61K 31/517; A61K 31/47

(52) U.S. Cl. 514/266.4; 514/313

(57) ABSTRACT

This invention provides compounds having the structure:

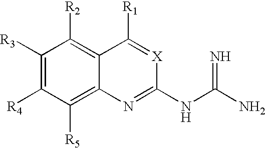

wherein X=CH, C(CH₃) or N; each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆; wherein Z is O or S; and wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl, wherein Q is OR₇, SR₇, N(R₇)₂ or aryl, wherein R₇ is H, alkyl, alkenyl, alkynyl, cycloalkyl, cycloalkenyl, aryl, wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring; and wherein each alkyl, alkenyl, alkynyl and alkoxy group is optionally substituted with a substituent independently selected from Rₐ, where Rₐ is 1) hydroxy, 2) C₁-C₁₀ alkoxy, 3) halogen, 4) nitro, 5) amino, 6) CF₃, or 7) carboxy, and each cycloalkyl group is optionally substituted with a substituent independently selected from R_{b}, where R_{b} is 1) a group selected from Rₐ, 2) C₁-C₇ alkyl, 3) C₂-C₇ alkenyl, 4) C₂-C₇ alkynyl or 5) cyclic C₁-C₁₀ alkyl, and each aryl is optionally substituted with R₁. This invention also provides methods of treating pain, urge incontinence; as well as methods of preparing the compounds.

Throughout this application, various publications are referenced within parentheses. Disclosures of these publications in their entireties are hereby incorporated by reference into this application to more fully describe the state of the art to which this invention pertains. Full bibliographic citations for these references may be found immediately preceding the claims.

### BACKGROUND OF THE INVENTION

NPFF is an octapeptide isolated from bovine brain in 1985 by Yang and coworkers (1) using antibodies to the molluscan neuropeptide FMRFamide (FMRFa). FMRFamide-like immunoreactivity was observed in rat brain, spinal cord, and pituitary, suggesting the existence of mammalian homologs of the FMRFa family of invertebrate peptides. The isolation of NPFF, named for its N- and C-terminal phenylalanines (also called F8Famide) and a second mammalian peptide, NPAF (also called A18Famide), confirmed the existence of a mammalian family of peptides sharing C-terminal sequence homology with FMRFa (1). Molecular cloning has revealed that NPFF and NPAF are encoded by the same gene and cleaved from a common precursor protein (2). Studies of the localization, radioligand binding, and function of NPFF-like peptides indicate they are neuromodulatory peptides whose effects are likely to be mediated by G protein-coupled receptors (4).

There are two known receptor subtypes for NPFF, NPFF-1 and NPFF-2 (3). Recently, two NPFF receptor subtypes (NPFF-1 and NPFF-2) were discovered and cloned from rat and human tissues (4). The localization of protein and mRNA for these two receptors indicates that they may have utility as targets for drugs to treat a variety of disorders including, but not limited to, disorders of electrolyte balance, diabetes, respiratory disorders, gastrointestinal disorders, depression, phobias, anxiety, mood disorders, cognition/memory disorders, obesity, pain, alertness/sedation, lower urinary tract disorders and cardiovascular indications.

NPFF is an endogenous modulator of opioid systems with effects on morphine analgesia, tolerance, and withdrawal (5, 6). NPFF appears to represent an endogenous “anti-opioid” system in the CNS, acting at specific high-affinity receptors that are distinct from opioid receptors (7, 8). Endogenous NPFF has been suggested to play a role in morphine tolerance: agonists of NPFF precipitate “morphine abstinence syndrome” (symptoms of morphine withdrawal) in morphine-dependent animals (9, 10), while antagonists and anti-NPFF IgG restore morphine sensitivity and ameliorate symptoms of withdrawal. NPFF has also been shown to participate in the regulation of pain threshold, showing both “anti-opiate” effects and analgesic effects, depending on the test system (5).

The ability of NPFF peptides to modulate the opioid system raised the possibility that NPFF interacts directly with opiate receptors. However, radioligand binding assays using a tyrosine-substituted NPFF analog [¹²⁵I]Y8Fa demonstrate that NPFF acts through specific high affinity binding sites distinct from opiate receptors (11-14) that are sensitive to inhibition by guanine nucleotides (15).

NPFF and related peptidic agonists exhibit direct analgesic activity in some animal models. NPFF has been shown to produce analgesia in the rat tail-flick and paw pressure models, upon intrathecal administration (16). Similarly, a NPFF-like peptide, SLAAPQRF-amide, isolated from rat brain and spinal cord (17), produces antinociceptive action in the tail-flick and paw pressure models (18). NPFF has also been observed to play a role in animal models of chronic pain. For example, NPFF has recently been shown to be involved in inflammatory pain (19) and neuropathic pain (20). Importantly, NPFF was shown to attenuate the allodynia associated with neuropathic pain, suggesting that it may be clinically useful in treating this condition. NPFF also has been shown to produce nighttime hyperasthesic analgesia in the tail-flick test upon i.c.v. administration in the rat (21). A peptidic NPFF analog, (D)Tyr¹, (NMe)Phe³— NPFF (1DMe, 1DMeY8Fa), which is partially protected against enzymatic degradation and also has high affinity for its receptors, shows long-lasting analgesic activity in the above models upon intrathecal administration (22, 23). In carrageenan inflammation, 5-10 nmol of 1DMe was effective against both thermal hyperalgesia and mechanical allodynia, and in a neuropathic pain model, 1DMe showed antiallodynic effects against cold allodynia (24). 1DMe also shows analgesic activity in the rat vocalization threshold upon intrathecal administration (25).

Recent studies in our laboratories have shown that NPFF also has peripheral effects. NPFF and related agonists show decrease in the contraction frequency of the rat bladder upon i.v. and i.t. administration (See PCT International Publication No. WO 00/18438). A potent NPFF agonist, PFRF-amide, has been shown to increase blood pressure and heart rate in rats (26). In addition, NPFF and related peptides have a number of other biological activities that may be therapeutically relevant including effects on feeding (27-29), psychotic behavior (30), nicotine addiction (31), and other cardiovascular functions (32, 33).

Effects on feeding behavior are further supported by findings that demonstrate NPFF-like immunoreactive neurons, as well as NPFF1 receptor mRNA, localize to the hypothalamus (3,5). The NPFF1-selective ligand, BIBP 3226, which is also a neuropeptide Y Y1 antagonist, blocks feeding through a nonspecific mechanism, not secondary to inhibition of Y1 (39). These data suggest that feeding behavior may be regulated through a NPFF1 receptor mechanism.

It is thus evident that NPFF agonists and/or antagonists have great potential as being therapeutically useful agents for the treatment of a diverse array of clinically relevant human disorders. NPFF agonists may have therapeutic potential, among others, for the treatment of pain, memory loss, circadian rhythm disorders, and micturition disorders. Cloned receptor subtypes of NPFF and the development of high-efficiency in vitro assays, both for binding and receptor activation, has aided the discovery and development of novel NPFF ligands. Moreover, it is practically possible to design a molecule that is an agonist at one NPFF subtype, and an antagonist at the other(s). This concept of a dual-acting molecule provides an attractive means of designing drugs that can treat multiple disorders.

There are no known nonpeptide agonists or antagonists of NPFF in the prior art. Described herein are quinazolino- and quinolino-guanidine containing compounds that may be used to treat an abnormality in a subject wherein the abnormality is, alleviated by increasing or decreasing the activity of a mammalian NPFF receptor which comprises administering to the subject an amount of a compound which is an antagonist or agonist of mammalian NPFF receptors to effect a treatment of the abnormality. The compounds of invention herein are the first known small molecule (non-peptide/non-peptoid) ligands (either antagonists or agonists)at the neuropeptide FF(NPFF) receptor(s).

### SUMMARY OF THE INVENTION

This invention provides a method of treating urge incontinence in a subject in need of such treatment comprising administering to the subject an effective amount of a compound having the structure:

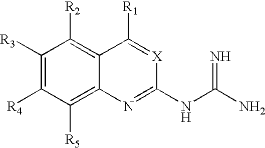

wherein X=CH, C(CH₃) or N;

wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆ —OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₂ and R₃ and the carbons to which, they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;

and wherein each alkyl, alkenyl, alkynyl and alkoxy group is optionally substituted with a substituent independently selected from Rₐ, where Rₐ is

1) hydroxy,

2) C₁-C₁₀ alkoxy,

3) halogen,

4) nitro,

5) amino,

6) CF₃, or

7) carboxy,

and each cycloalkyl group is optionally substituted with a substituent independently selected from R_{b}, where R_{b} is

1) a group selected from Rₐ,

2) C₁-C₇ alkyl,

3) C₂-C₇ alkenyl,

4) C₂-C₇ alkynyl or

5) cyclic C₁-C₁₀ alkyl,

and each aryl is optionally substituted with R₁,

to thus treat the urge incontinence in the subject.

This invention also provides a method of treating pain in a subject in need of such treatment comprising administering to the subject an effective amount of the aforementioned compound.

### BRIEF DESCRIPTION OF THE FIGURES

FIG. 1: Shows the correlation between the binding affinities at human and rat recombinant neuropeptide FF receptors. The binding affinities (pKi values) for 18 compounds were tested at rat NPFF receptors and plotted against the pKi values for the same 18 compounds tested at human NPFF2 receptors. A slope value of 0.83 was obtained for rat NPFF1 vs. human NPFF1 and a slope value of 0.75 was obtained for rat NPFF2 vs. human NPFF2, both slope values of which indicate a positive correlation.

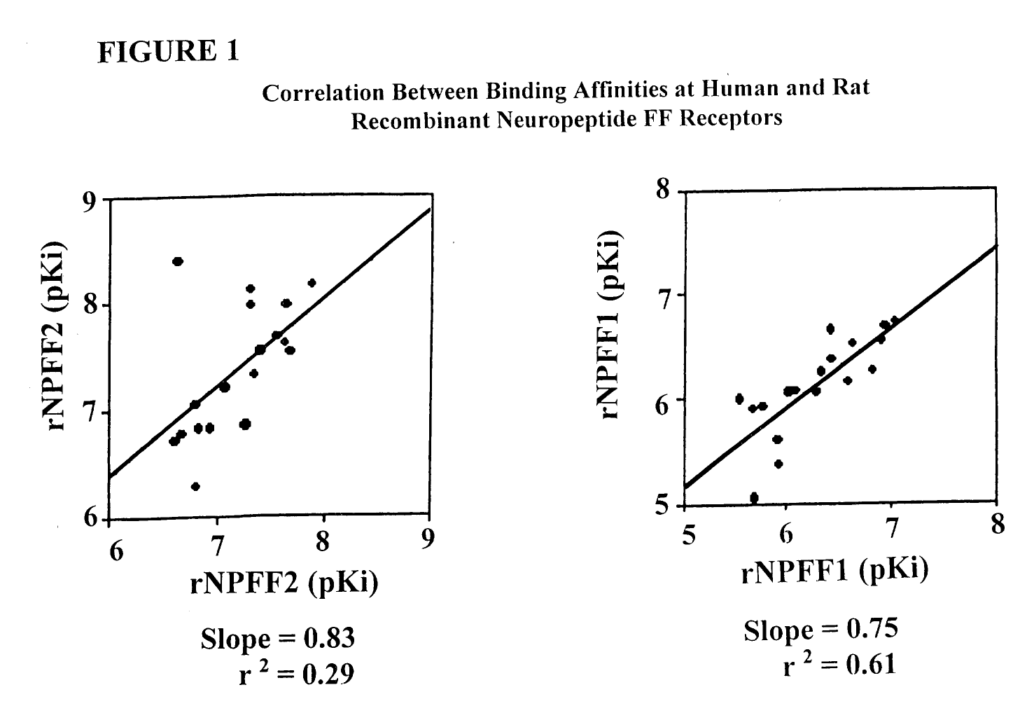

FIG. 2: Shows the effect of compound (4006) on bladder activity in the anesthetized rat. Rhythmic elevations in bladder pressure, resulting from distension induced contractions, were unaffected by the i.v. administration of physiological saline. In contrast, the NPFF receptor ligand compound (4006) produced an immediate inhibition of bladder activity, which persisted for 12 min.

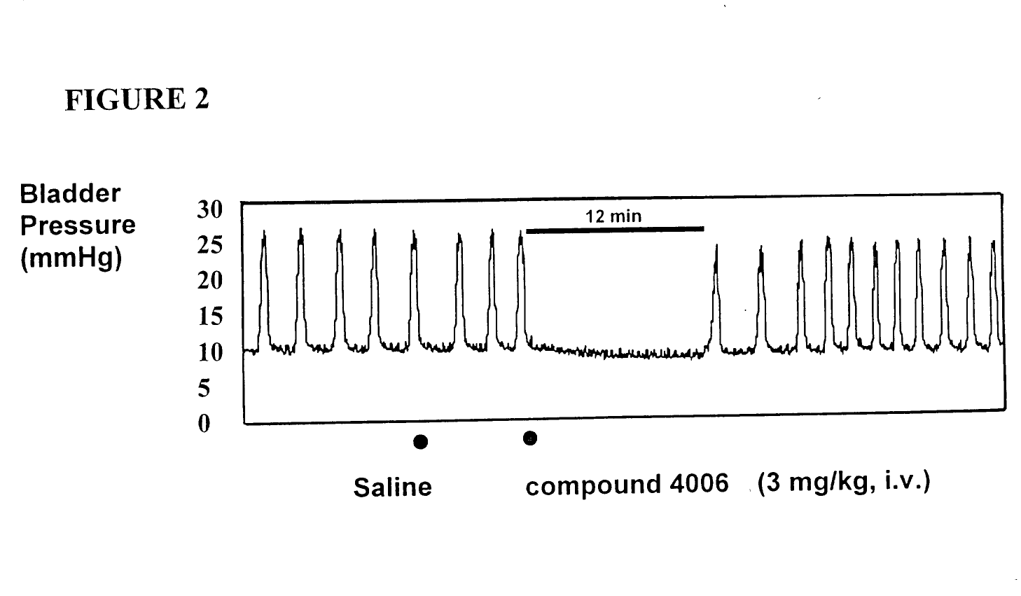

FIG. 3: Shows the effect of compound (4005) on bladder activity in the anesthetized rat. Rhythmic elevations in bladder pressure, resulting from distension induced contractions, were unaffected by the i.v. administration of physiological saline. In contrast, the NPFF receptor ligand compound (4005) produced an immediate inhibition of bladder activity, which persisted for 35 min.

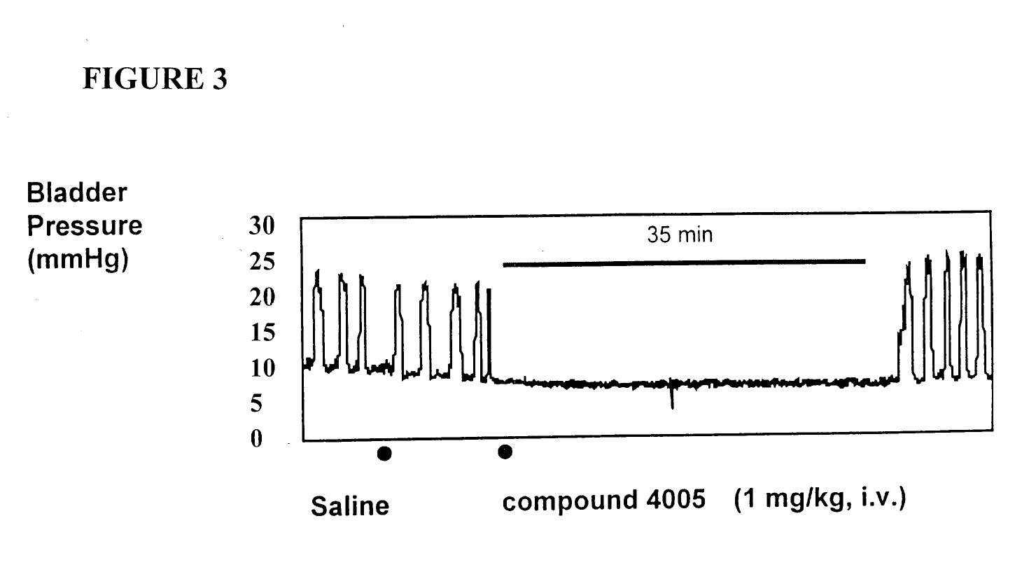

### DETAILED DESCRIPTION OF THE INVENTION

The present invention provides a method of treating urge incontinence in a subject in need of such treatment comprising administering to the subject an effective amount of a compound having the structure:

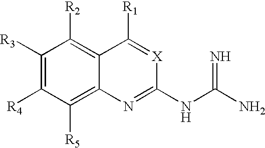

wherein X=CH, C(CH₃) or N;

wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆ —OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;

and wherein each alkyl, alkenyl, alkynyl and alkoxy group is optionally substituted with a substituent independently selected from Rₐ, where Rₐ is

1) hydroxy,

2) C₁-C₁₀ alkoxy,

3) halogen,

4) nitro,

5) amino,

6) CF₃, or

7) carboxy,

and each cycloalkyl group is optionally substituted with a substituent independently selected from R_{b}, where R_{b} is

1) a group selected from Rₐ,

2) C₁-C₇ alkyl,

3) C₂-C₇ alkenyl,

4) C₂-C₇ alkynyl or

5) cyclic C₁-C₁₀ alkyl,

and each aryl is optionally substituted with R₁,

to thus treat the urge incontinence in the subject.

This invention also provides a method of treating pain in a subject in need of such treatment comprising administering to the subject an effective amount of any of the aforementioned compounds.

In one embodiment of the aforementioned method, wherein R₁ may be methyl or ethyl;

wherein R₂ is H or fused benzene;

wherein R₃ is H, methyl, ethyl, propyl, tert-butyl, octyl, cyclohexyl, phenyl, hydroxy, methoxy, butoxy, pentoxy, phenoxy, benzoxy, trifluoromethyl ether, methylbenzene ether, 5-phenoxypentyloxy, 4-Hydroxypentyl, Cl, Br, F, or wherein R₂ and R₃ and the carbons to which they are attached form a fused benzene, fused 5,6-cyclohexenyl, fused cyclopentyl, or fused 2,3-furyl; and

wherein R₄ is H, methyl, ethyl, isopropyl, tert-butyl, 1-hydroxyethyl, ethoxy, butoxy, isopropoxy, phenoxy, benzyloxy, trifluoromethyl ether, Br, F, or wherein R₃ and R₄ and the carbons to which they are attached form a fused benzene, fused 5,6-cyclohexenyl, fused cyclopentyl, or fused 2,3-furyl.

In another embodiment of the aforementioned method, wherein R₁ is methyl or ethyl;

wherein R₂ is H;

wherein R₃ is propyl, octyl, cyclohexyl, phenyl, hydroxy, methoxy, butoxy, pentoxy, phenoxy, benzoxy, trifluoromethyl ether, methylbenzene ether, 4-Hydroxypentyl, Cl, Br, F, or wherein R₂ and R₃ and the carbons to which they are attached form fused 5,6-cyclohexenyl, fused cyclopentyl, or fused 2,3-furyl; and

wherein R₄ is H, methyl, ethyl, isopropyl, tert-butyl, 1-hydroxy ethyl, ethoxy, butoxy, isopropoxy, phenyl, Br, F, or wherein R₃ and R₄ and the carbons to which they are attached form a fused 5,6-cyclohexenyl, fused cyclopentyl, or fused 2,3-furyl.

In another embodiment of the aforementioned method, wherein R₁ is methyl or ethyl;

wherein R₂ is H;

wherein R₃ is cyclohexyl, benzoxy, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl; and

wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

In another embodiment of the aforementioned method, wherein R₁ is methyl or ethyl;

wherein R₂ is H;

wherein R₃ is cyclohexyl, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl;

wherein R₄ is H, l-hydroxyethyl, trifluoromethyl ether, or wherein R₃ and R₄ and the carbons to which they are attached form fused 5,6-cyclohexenyl, fused cyclopentyl, or fused 2,3-furyl.

In another embodiment of the aforementioned method, a compound having the structure:

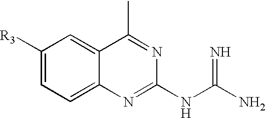

wherein R₃ is H, straight chained or branched C₁-C₇ alkyl or aryl.

In another embodiment of the aforementioned method, wherein R₃ is butyl, sec-butyl, pentyl, hexyl, heptyl, or benzyl.

In another embodiment of the aforementioned method, wherein R₃ is butyl, sec-butyl, hexyl, heptyl, or benzyl.

In another embodiment of the aforementioned method, the compound has the structure:

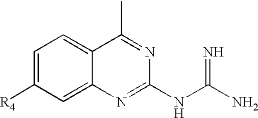

wherein R₄ is H, straight chained or branched C₁-C₇ alkyl.

In another embodiment of the aforementioned method, wherein R₄ is H, or methyl.

In another embodiment of the aforementioned method, the compound has the structure:

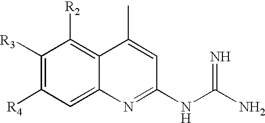

wherein R₂ is H or methyl;

wherein R₃ is H, straight chained or branched C₁-C₇ alkyl, aryl, alkoxy or halogen, or wherein R₂ and R₃ and the carbons to which they are attached form a fused aryl; and

wherein R₄ is H, methyl or halogen.

In another embodiment of the aforementioned method, wherein R₂ is H, methyl;

wherein R₃ is H, Cl, methyl, ethyl, methoxy, phenyl or wherein R₂ and R₃ and the carbons to which they are attached form fused benzene; and

wherein R₄ is H, methyl or F.

In another embodiment of the aforementioned method, the compound has the structure:

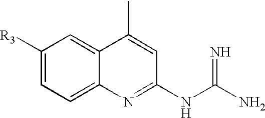

wherein R₃ is H, straight chained or branched C₁-C₇ alkyl.

In another embodiment of the aforementioned method, wherein R₃ is butyl, pentyl or hexyl.

In another embodiment of the aforementioned method, the compound has the structure:

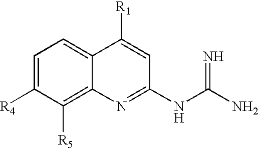

wherein R₁ is H, straight chained or branched C₁-C₇ alkyl; and

wherein each R₄ and R₅ is independently H or straight chained or branched C₁-C₇ alkyl.

In another embodiment of the aforementioned method, wherein R₁ is methyl or ethyl; and

wherein each R₄ and R₅ is independently H or methyl.

In another embodiment of the aforementioned method, the compound has the structure:

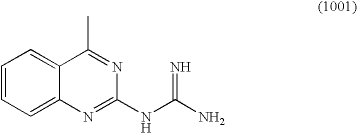

In another embodiment of the aforementioned method, the compound has the structure:

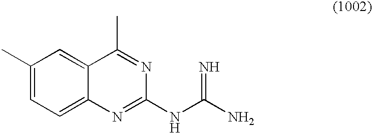

In another embodiment of the aforementioned method, the compound has the structure:

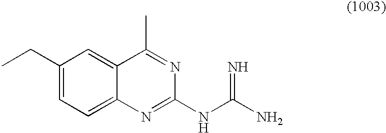

In another embodiment of the aforementioned method, the compound has the structure:

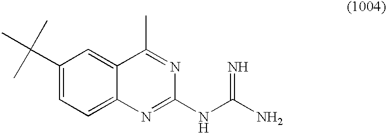

In another embodiment of the aforementioned method, the compound has the structure:

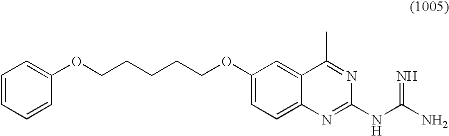

In another embodiment of the aforementioned method, the compound has the structure:

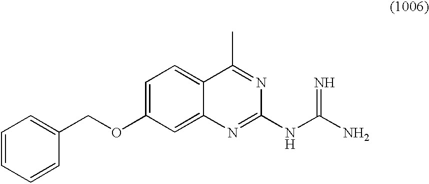

In another embodiment of the aforementioned method, the compound has the structure:

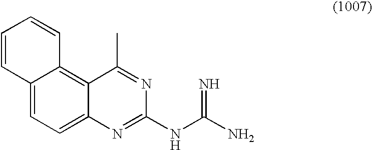

In another embodiment of the aforementioned method, the compound has the structure:

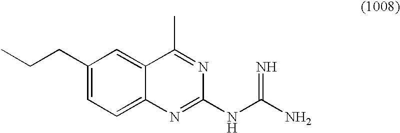

In another embodiment of the aforementioned method, the compound has the structure:

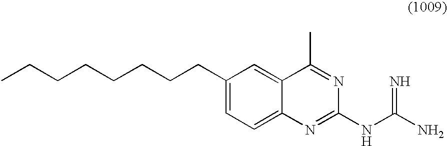

In another embodiment of the aforementioned method, the compound has the structure:

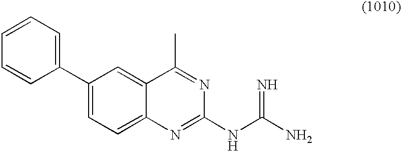

In another embodiment of the aforementioned method, the compound has the structure:

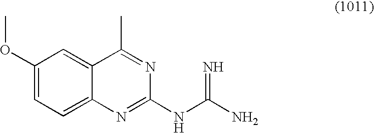

In another embodiment of the aforementioned method, the compound has the structure:

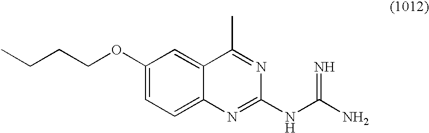

In another embodiment of the aforementioned method, the compound has the structure:

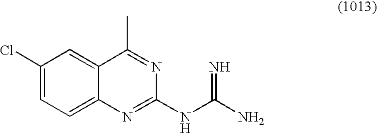

In another embodiment of the aforementioned method, the compound has the structure:

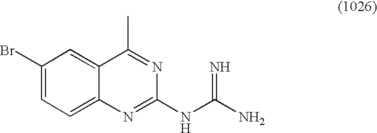

In another embodiment of the aforementioned method, the compound has the structure:

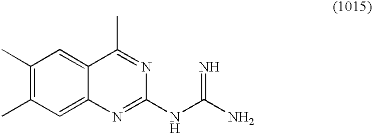

In another embodiment of the aforementioned method, the compound has the structure:

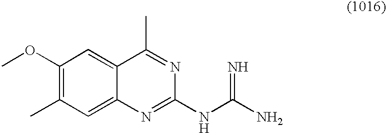

In another embodiment of the aforementioned method, the compound has the structure:

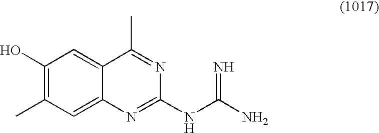

In another embodiment of the aforementioned method, the compound has the structure:

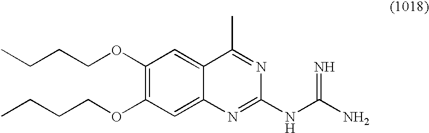

In another embodiment of the aforementioned method, the compound has the structure:

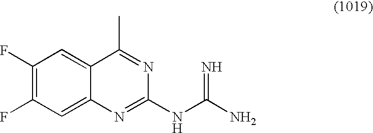

In another embodiment of the aforementioned method, the compound has the structure:

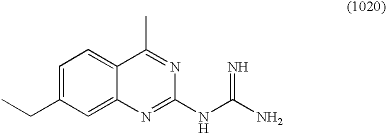

In another embodiment of the aforementioned method, the compound has the structure:

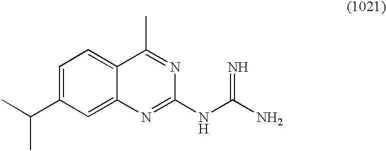

In another embodiment of the aforementioned method, wherein the compound has the structure:

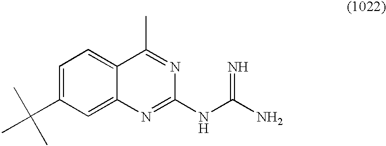

In another embodiment of the aforementioned method, the compound has the structure:

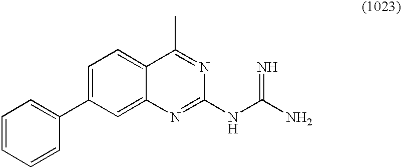

In another embodiment of the aforementioned method, the compound has the structure:

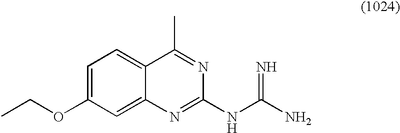

In another embodiment of the aforementioned method, the compound has the structure:

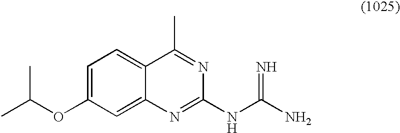

In another embodiment of the aforementioned method, the compound has the structure:

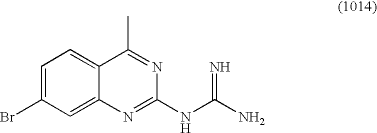

In another embodiment of the aforementioned method, the compound has the structure:

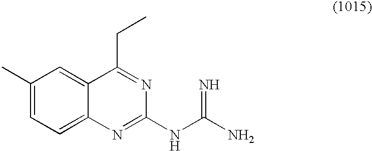

In another embodiment of the aforementioned method, the compound has the structure:

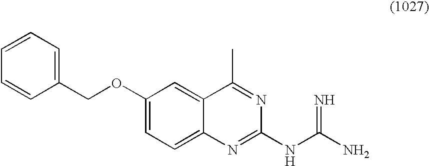

In another embodiment of the aforementioned method, the compound has the structure:

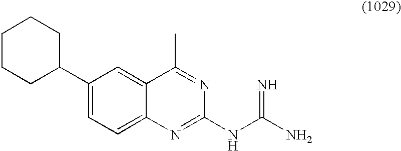

In another embodiment of the aforementioned method, the compound has the structure:

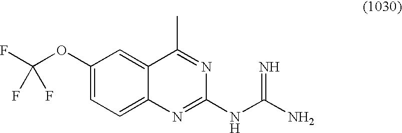

In another embodiment of the aforementioned method, the compound has the structure:

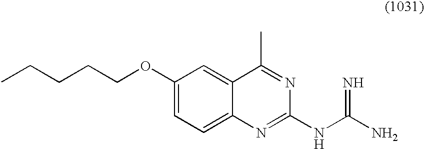

In another embodiment of the aforementioned method, the compound has the structure:

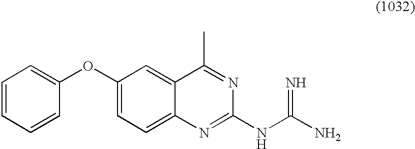

In a further embodiment of the above described method, wherein the compound has the structure:

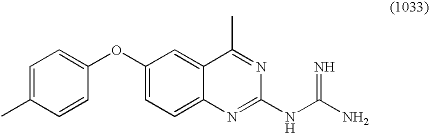

In another embodiment of the aforementioned method, the compound has the structure:

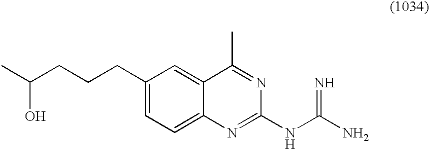

In another embodiment of the aforementioned method, the compound has the structure:

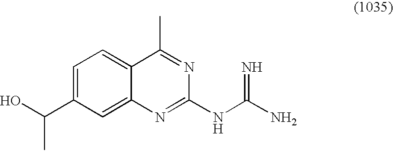

In another embodiment of the aforementioned method, the compound has the structure:

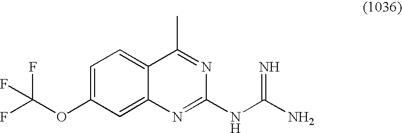

In another embodiment of the aforementioned method, the compound has the structure:

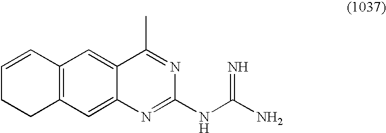

In another embodiment of the aforementioned method, the compound has the structure:

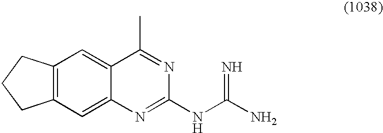

In another embodiment of the aforementioned method, the compound has the structure:

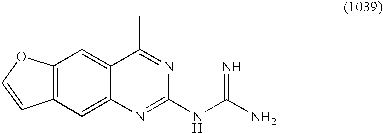

In another embodiment of the aforementioned method, the compound has the structure:

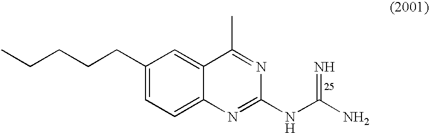

In another embodiment of the aforementioned method, the compound has the structure:

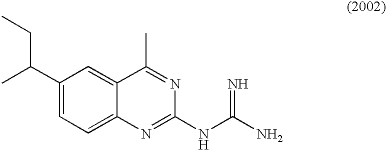

In another embodiment of the aforementioned method, the compound has the structure:

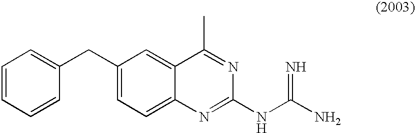

In another embodiment of the aforementioned method, the compound has the structure:

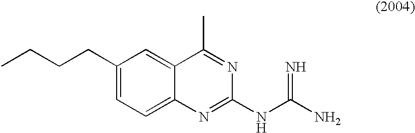

In another embodiment of the aforementioned method, compound has the structure:

In a further embodiment of the above described method, wherein the compound has the structure:

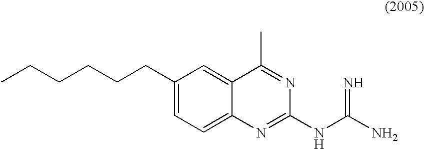

In another embodiment of the aforementioned method, the compound has the structure:

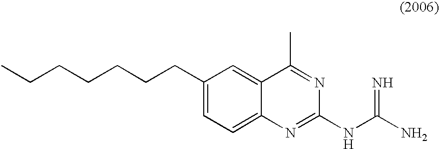

In another embodiment of the aforementioned method, the compound has the structure:

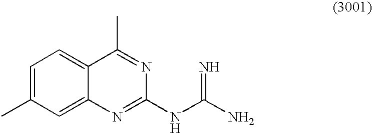

In another embodiment of the aforementioned method, the compound has the structure:

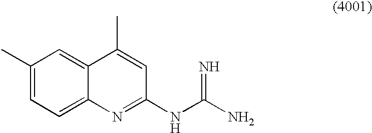

In another embodiment of the aforementioned method, the compound has the structure:

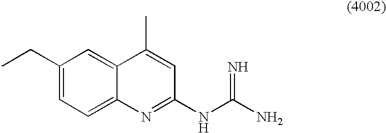

In another embodiment of the aforementioned method, the compound has the structure:

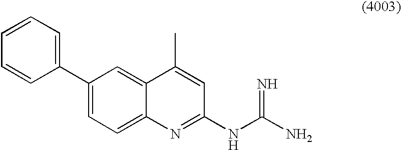

In another embodiment of the aforementioned method, the compound has the structure:

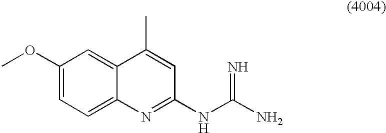

In another embodiment of the aforementioned method, the compound has the structure:

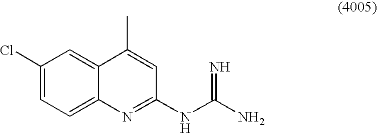

In another embodiment of the aforementioned method, the compound has the structure:

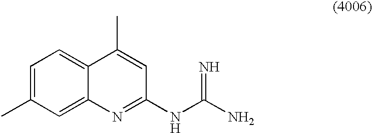

In another embodiment of the aforementioned method, the compound has the structure:

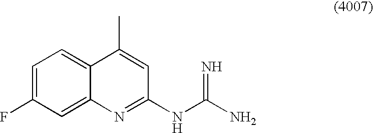

In another embodiment of the aforementioned method, the compound has the structure:

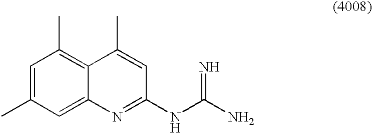

In another embodiment of the aforementioned method, the compound has the structure:

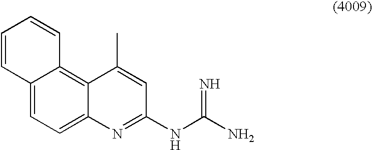

In another embodiment of the aforementioned method, the compound has the structure:

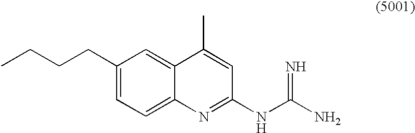

In another embodiment of the aforementioned method, the compound has the structure:

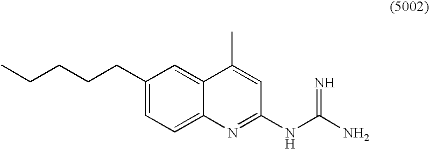

In another embodiment of the aforementioned method, the compound has the structure:

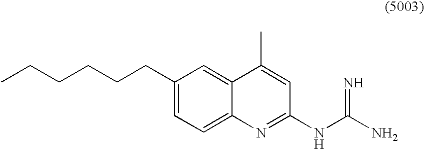

In another embodiment of the aforementioned method, the compound has the structure:

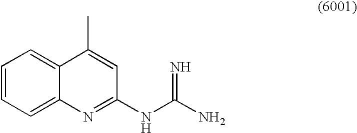

In another embodiment of the aforementioned method, the compound has the structure:

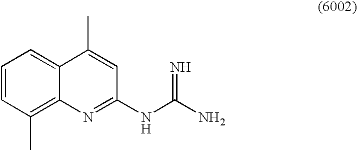

In another embodiment of the aforementioned method, the compound has the structure:

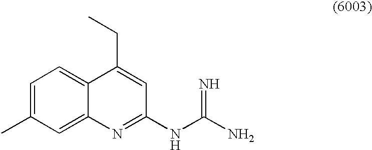

This invention further includes a compound having the structure:

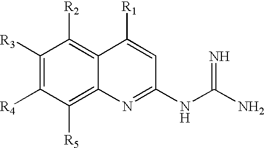

wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)2, —OC(═Z)R₆, —C(═Z)OR₆ —OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;

and wherein each alkyl, alkenyl, alkynyl and alkoxy group is optionally substituted with a substituent independently selected from Rₐ, where Rₐ is

1) hydroxy,

2) C₁-C₁₀ alkoxy,

3) halogen,

4) nitro,

5) amino,

6) CF₃, or

7) carboxy,

and each cycloalkyl group is optionally substituted with a substituent independently selected from R_{b}, where R_{b} is

1) a group selected from Rₐ,

2) C₁-C₇ alkyl,

3) C₂-C₇ alkenyl,

4) C₂-C₇ alkynyl or

5) cyclic C₁-C₁₀ alkyl,

and each aryl is optionally substituted with R₁.

The present invention further includes a compound having the structure:

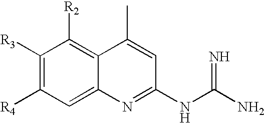

wherein R₂ is H or methyl;

wherein R₃ is H, straight chained or branched C₁-C₇ alkyl, aryl, alkoxy or halogen, or wherein R₂ and R₃ and the carbons to which they are attached form a fused aryl; and

wherein R₄ is H, methyl or halogen.

The present invention further includes the aforementioned compound wherein R₂ is H, methyl;

wherein R₃ is H, Cl, methyl, ethyl, methoxy, phenyl or wherein R₂ and R₃ and the carbons to which they are attached form fused benzene; and

wherein R₄ is H, methyl or F.

The present invention further includes a compound having the structure:

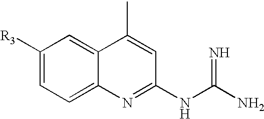

wherein R₃ is H, straight chained or branched C₁-C₇ alkyl.

The present invention further includes the aforementioned compound wherein R₃ is propyl, pentyl or hexyl.

This invention further includes a compound having the structure:

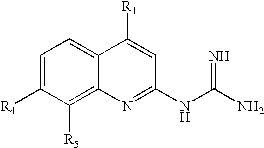

wherein R₁ is H, straight chained or branched C₁-C₇ alkyl; and

wherein each R₄ and R₅ is independently H or straight chained or branched C₁-C₇ alkyl.

This invention further includes the aforementioned compound wherein R₁ is methyl or ethyl; and

wherein each R₄ and R₅ is independently H or methyl.

This invention also includes a compound having the structure:

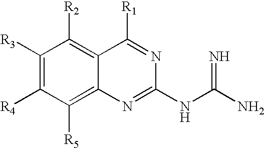

wherein each of R₁, R₂, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆ —OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₃ is straight chained C₃, C₄, C₆ or C₇ alkyl or branched C₅-C₇ alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆ —OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₂ and R₃ and the carbons to which they are attached form a fused cyclic alkyl or heterocyclic alkyl ring; or wherein R₃ and R₄ and the carbons to which they are attached form a fused aryl, heteroaryl, cyclic alkyl or heterocyclic alkyl ring; and

and wherein each alkyl, alkenyl, alkynyl and alkoxy group is optionally substituted with a substituent independently selected from Rₐ, where Rₐ is

1) hydroxy,

2) C₁-C₁₀ alkoxy,

3) halogen,

4) nitro,

5) amino,

6) CF₃, or

7) carboxy,

and each cycloalkyl group is optionally substituted with a substituent independently selected from R_{b}, where R_{b} is

1) a group selected from Rₐ,

2) C₁-C₇ alkyl,

3) C₂-C₇ alkenyl,

4) C₂-C₇ alkynyl or

5) cyclic C₁-C₁₀ alkyl,

and each aryl is optionally substituted with R₁.

This invention also includes the compound having the structure:

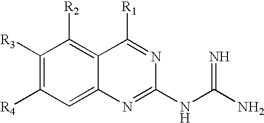

herein R₁ is H, straight chained or branched C₁-C₇ alkyl;

wherein R₂ is H, straight chained or branched C₁-C₇ alkyl or fused aryl;

wherein R₃ is straight chained C₃, C₄, C₆ or C₇ alkyl or branched C₅-C₇ alkyl, cycloalkyl, substituted or unsubstituted aryl, hydroxyl, straight chained or branched alkoxy, halogenated ether, or halogen;

wherein R₄ is H, branched C₁-C₇ alkyl, aryl, straight chained or branched alkoxy or halogen; or wherein R₂ and R₃ and the carbons to which they are attached form a fused C₃-C₆ cyclic alkyl or heterocyclic alkyl ring; or wherein R₃ and R₄ and the carbons to which they are attached form a fused C₆-C₇ aryl or heteroaryl ring, a fused C₃-C₆ cyclic alkyl or heterocyclic alkyl ring.

This invention further includes the aforementioned compound wherein R₁ is methyl or ethyl;

wherein R₂ is H or fused benzene;

wherein R₃ is cyclohexyl, phenyl, hydroxy, methoxy, butoxy, pentoxy, phenoxy, benzoxy, trifluoromethyl ether, methylbenzene ether, 4-Hydroxypentyl, Cl, Br, F, or wherein R₂ and R₃ and the carbons to which they are attached form fused 5,6-cyclohexenyl, fused cyclopentyl, or fused 2,3-furyl; and

wherein R₄ is H, isopropyl, tert-butyl, 1-hydroxyethyl, ethoxy, butoxy, isopropoxy, phenyl, Br, F, or wherein R₃ and R₄ and the carbons to which they are attached form fused 5,6-cyclohexenyl, fused cyclopentyl, or fused 2,3-furyl.

This invention further includes the aforementioned compound wherein R₁ is methyl or ethyl;

wherein R₂ is H or fused benzene;

wherein R₃ is cyclohexyl, benzoxy, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl; and

wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

This invention further includes the aforementioned compound wherein R₁ is methyl or ethyl;

wherein R₂ is H or fused benzene;

wherein R₃ is cyclohexyl, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl;

wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

This invention further includes the compound having the structrue:

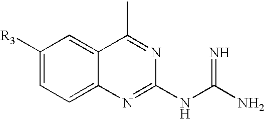

wherein R₃ is straight chained C₃, C₄, C₆ or C₇ alkyl or branched C₅-C₇ alkyl or aryl.

This invention further includes the aforementioned compound wherein R₃ is butyl, hexyl, heptyl, or benzyl.

This invention further includes the compound having the structure:

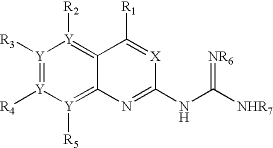

wherein X=CH, C(CH₃) or N;

wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;

wherein Z is O or S; and

wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,

wherein Q is OR₇, SR₇, N(R₇)₂ or aryl,

wherein R₇ is H, alkyl, alkenyl, alkynyl, cycloalkyl, cycloalkenyl, aryl,

wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring; and wherein each alkyl, alkenyl, alkynyl and alkoxy group is optionally substituted with a substituent independently selected from Rₐ, where Rₐ is

1) hydroxy,

2) C₁-C₁₀ alkoxy,

3) halogen,

4) nitro,

5) amino,

6) CF₃, or

7) carboxy,

and each cycloalkyl group is optionally substituted with a substituent independently selected from R_{b}, where R_{b} is

1) a group selected from Rₐ,

2) C₁-C₇ alkyl,

3) C₂-C₇ alkenyl,

4) C₂-C₇ alkynyl or

5) cyclic C₁-C₁₀ alkyl,

and each aryl is optionally substituted with R₁, and

wherein each R₆ and R₇ is independently acetate, formate, phosphate ester, dimethylglycine ester, aminoalkylbenzyl ester, aminoalkyl ester and carboxyalkyl ester.

This invention further includes the aforementioned compound wherein R₆ and R₇ is independently acetyl or acyl.

This invention provides a pharmaceutical composition comprising any of the aforementioned compounds together with a pharmaceutically acceptable carrier.

This invention further provides a method of preparing a pharmaceutical composition comprising mixing the compound of any of the aforementioned compounds with a pharmaceutical acceptable carrier.

This invention further provides a compound which is converted in vivo to the compound of any of the aforementioned compounds.

This invention further provides a compound which is a metabolite of the compound of any of the aforementioned compounds

This invention further provides a salt of the compound of any of the aforementioned compounds.

For certain compounds, enantiomers, diastereomers, double bond stereoisomers and double bond regioisomers exist. This invention contemplates racemic mixtures as well as isolated enantiomers, double bond stereoisomers, double bond regioisomers and diastereomers.

The invention provides for each pure stereoisomer of any of the compounds described herein. Such stereoisomers may include enantiomers, disastereomers, or E or Z alkene isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) Or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J).

In addition the compounds of the present invention may be present as enatiomers, diasteriomers, isomers or two or more of the compounds may be present to form a racemic or diastereomeric mixture.

The compounds of the present invention are preferably 80% pure, more preferably 90% pure, and most preferably 95% pure.

As used herein, the term aryl is used to include phenyl, benzyl, or naphthyl, and the term hereroaryl is used to include pyrazinyl, imidazolyl, imidazolinyl, indolyl, benzimidazolyl, benzfuranyl, pyrimidinyl, benzothiophenyl, isoquinolyl, or quinolyl. The term arylalkyl is used to designate an C1-C6 alkyl chain substituted with an aryl group and the term heteroarylalkyl is used to designate a C1-C6 alkyl chain substituted with a heteroaryl group.

In the present invention, the term “heteroaryl” is used to include five and six membered unsaturated rings that may contain one or more oxygen, sulfur, or nitrogen atoms. Examples of heteroaryl groups include, but are not limited to, furanyl, thienyl, pyrroyl, oxazolyl, thiasolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, and triazinyl.

In addition the term “heteroaryl” is used to include fused bicyclic ring systems that may contain one or more heteroataoms such as oxygen, sulfur and nitrogen. Examples of such heteroaryl groups include, but are not limited to, indolizinyl, indolyl, isoindolyl, benzo[b]furanyl, benzo[b]thiophenyl, indazolyl, benzimidazolyl, purinyl, benaoxazolyl, benzisoxazolyl, benzo[b]thiazolyl, imidazo[2,1-b]thiazolyl, cinnolinyl, quinasolinyl, quinoxalinyl, 1,8-naphthyridinyl, pteridinyl, quinolinyl, isoquinolinyl, phthalimidyl and 2,1,3-benzothiazolyl.

Heterocyclic is defined as a 3 to 10 atom-ring containing at least one saturated bond and containing in any position one or more of the following atoms: N,O,S. Examples of heterocyclic rings include but are not limited to tetrahydrofuran, dihydrofuran, tetrahydropyran, kihydropyran piperidine, dihydropiperidine, pyrrolidine, dihydropyrrolidine dioxane, piperazin.

The compounds of invention herein are the first known small molecule (non-peptide/non-peptoid) ligands (either antagonists or agonists)at the neuropeptide FF(NPFF) receptor(s).

In separate embodiments, the abnormality is a lower urinary tract disorder such as interstitial cystitis or urinary incontinence such as urge incontinence or stress incontinence particularly urge incontinence, a regulation of a steroid hormone disorder, an epinephrine release disorder, a gastrointestinal disorder, irritable bowel syndrome, a cardiovascular disorder, an electrolyte balance disorder, diuresis, hypertension, hypotension, diabetes, hypoglycemia, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a serotonergic function disorder, an olfaction disorder, a sympathetic innervation disorder, an affective disorder, pain, psychotic behavior, morphine tolerance, nicotine addiction, opiate addiction, or migraine.

As used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

The formulations of the present invention can be solutions, suspensions, emulsions, syrups, elixirs, capsules, tablets, and the like. The compositions may contain a suitable carrier, diluent, or excipient, such as sterile water, physiological saline, glucose, or the like. Moreover, the formulations can also be lyophilized, and/or may contain auxiliary substances, such as wetting or emulsifying agents, pH buffering agents, adjuvants, gelling or viscosity enhancing additives, preservatives, flavoring agents, colors, and the like, depending upon the route of administration and the preparation desired Standard texts, such as “Remington's Pharmaceutical Science”, 17th Ed., 1985, incorporated herein by reference, may be consulted to prepare suitable preparations, without undue experimentation.

The formulations can include powdered carriers, such as lactose, sucrose, mannitol, starch, cellulose derivatives, magnesium stearate, stearic acid, and the like. Further, tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. The formulations can also contain coloring and flavoring to enhance patient acceptance. The formulations can also include any of disintegrants, lubricants, plasticizers, colorants, and dosing vehicles.

In general, water, a suitable oil, saline, aqueous dextrose (glucose), and related sugar solutions and glycols such as propylene glycol or polyethylene glycols such as propylene glycol or polyethylene glycols are suitable carriers for parenteral solutions. Solutions for parenteral administration contain preferably a water soluble salt of the active ingredient, suitable stabilizing agents, and, if necessary, buffer substances.

Antioxidants such as, for example, sodium bisulfate, sodium sulfite, citric acid and its salts, sodium EDTA, ascorbic acid, and the like can be used either alone or in combination with other suitable antioxidants or stabilizing agents typically employed in the pharmaceutical compositions. In addition, parenteral solutions can contain preservatives, such as, for example, benzalkonium chloride, methyl- or propyl-paraben, chlorobutanol and the like.

The present invention includes within its scope prodrugs of the compounds of this inventions. In general, such prodrugs will be functional derivatives of the compounds of the invention which are readily convertible in vivo into the required compound.

Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985, the content of which is incorporated into the subject decription by reference.

Included in this invention are pharmaceutically acceptable salts and complexes of all of the compounds described herein. The salts include, but are not limited to, the following acids and bases: Inorganic acids which include hydrochloric acid, hydrofluoric acid, hydrobromic acid, hydroiodic acid, sulfuric acid, and boric acid; organic acids which include acetic acid, trifluoroacetic acid, formic acid, oxalic acid, malonic acid, succinic acid, fumaric acid, tartaric acid, maleic acid, citric acid, methanesulfonic acid, trifluoromethanesulfonic acid, benzoic acid, glycolic acid, lactic acid, and mandelic acid; inorganic bases include ammonia and hydrazine; and organic bases which include methylamine, ethylamine, hydroxyethylamine, propylamine, dimethylamine, diethylamine, trimethylamine, triethylamine, ethylenediamine, hydroethylamine, morpholine, piperazine, and guanidine.

This invention further provides for the hydrates and polymorphs of all of the compounds described herein.

The present invention further includes metabolites of the compounds of the present invention. Metabolites include active species produced upon introduction of compounds of this invention into the biological milieu.

One skilled in the art will readily appreciate that appropriate biological assays will be used to determine the therapeutic potential of the claimed compounds for treating the above noted disorders.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

I. Synthesis of Chemical Compounds

General Method

All reactions were performed under an inert atmosphere (Argon) and the reagents, neat or in appropriate solvents, were transferred to the reaction vessel via syringe and cannula techniques. The parallel synthesis reaction arrays were performed in vials (without an inert atmosphere) using J-KEM heating shakers (Saint Louis, Mo.). Anhydrous solvents (i.e. tetrahydrofuran, toluene and 1-methyl-2-pyrrolidinone) were purchased from Aldrich Chemical Company (Milwaukee, Wis.) and used as received. The compounds described in this patent were named using ACD/Name program (version 2.51, Advanced Chemistry Development Inc., Toronto, Ontario, M5H2L3, Canada). ¹H and ¹³C spectra were recorded at 300 and 75 MHz (QE-300 Plus by Bruker Instruments, Billerica, Mass.). Chemical shifts are reported in parts per million (ppm) and referenced with respect to the residual (i.e. CHCl₃, CH₃OH) proton of the deuterated solvent. Splitting patterns are designated as s=singlet; d=doublet; t=triplet; q=quartet; p=quintet; sextet; septet; broad=br; m=multiplet. Elemental analyses were performed by Robertson Microlit Laboratories, Inc. (Madison, N.J.) Low-resolution electrospray mass spectra (ESMS) were measured and MH⁺ is reported. Thin-layer chromatography (TLC) was carried out on glass plates precoated with silica gel 60 F₂₅₄ (0.25 mm, EM Separations Tech.). Preparative TLC was carried out on glass sheets precoated with silica gel GF (2 mm, Analtech, Newark, Del.). Flash column chromatography was performed on Merck silica gel 60 (230-400 mesh).

The following (Scheme 1) is a representative synthetic scheme for the synthesis of quinazolino-guanidines (32, 33a, b).

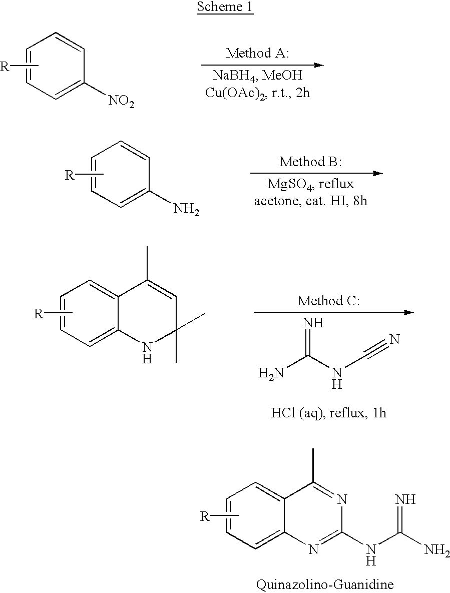

An alternative route (34) for the synthesis of quinazolino-guanidines is illustrated below (Scheme 2).

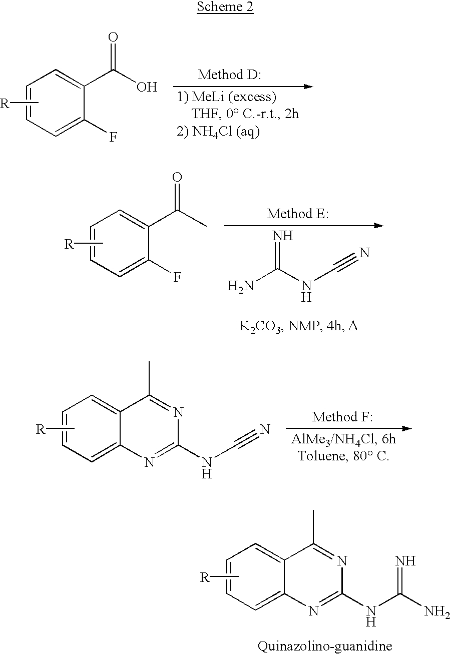

The following (Scheme 3) is a representative synthetic scheme for the synthesis of quinolino-guanidines (35).

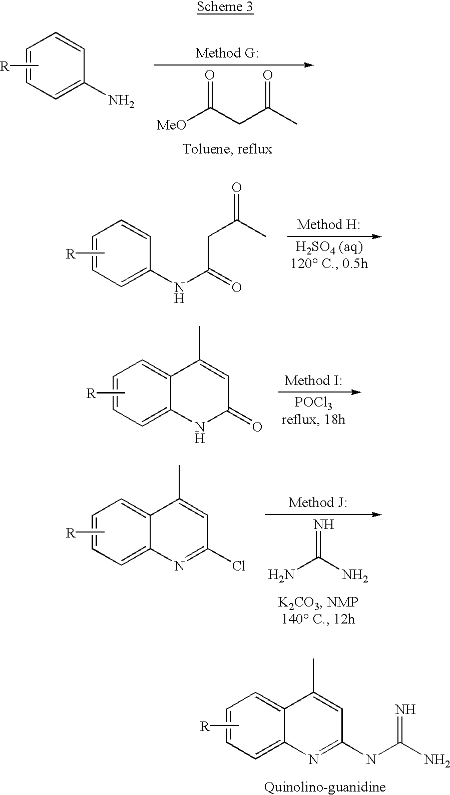

### Example 1

The following is a representative example of Methods A-C in Scheme 1 for the synthesis of N-(6,7-dibutoxy-4-methyl-2-quinazolinyl)guanidine (Compound 1018).

Method A (Ref #1)

In a flask equipped with a magnetic stirrer, 1,2-dibutoxy-4-nitrobenzene (500 mg, 1.87 mmol) was dissolved in methyl alcohol (23 mL). To this stirring solution was added a saturated aqueous solution of copper (II) acetate (7.5 mL) followed by sodium borohydride (779 mg, 20.6 mmol) added in several small portions so as keep the reaction solution from bumping. After all the sodium borohydride had been added, the solution was allowed to stir at room temperature (r.t.) for an additional 2 h. Brine (100 mL) was added followed by extraction of the aqueous phase with ethyl ether (2×) in a separatory funnel. The combined ethereal extracts were washed with saturated aqueous sodium bicarbonate. The ether was evaporated and the crude material further purified by silica column chromatography eluting with 50% ethyl acetate in hexane (Rf=0.20). The fractions were combined and solvent evaporated to afford 323 mg (73% yield) of 3,4-dibutoxyaniline.

Method B (Ref #2)

In a flask equipped with a magnetic stirrer, 3,4-dibutoxyaniline (323 mg, 1.36 mmol) was dissolved in acetone (2.3 mL). To this stirring solution was added magnesium sulfate (5.0 eq, 819 mg, 6.80 mmol), tert-butylcatechol (0.03 eq, 7 mg, 0.04 mmol) and iodine (0.05 eq, 17 mg, 0.07 mmol), in that order. The solution was refluxed for 8 h. Upon cooling to r.t., the solution was filtered and the residue further washed with methyl alcohol. The residue was purified by silica column chromatography eluting with 25% ethyl acetate in hexane to afford 230 mg (53% yield) of 6,7-dibutoxy-2,2,4-trimethyl-1,2-dihydroquinoline.

Method C

In a flask equipped with a magnetic stirrer, 6,7-dibutoxy-2,2,4-trimethyl-1,2-dihydroquinoline (230 mg, 0.72 mmol) was dissolved in 0.5 mL of a solution made up of 0.1 mL of 37% aqueous hydrochloric acid+0.4 mL of water. This solution was refluxed for 1 h. Upon cooling to r.t., 1.5 mL of a 2.0 M ammonia solution in methyl alcohol was added followed by evaporation of the solvent. Purification via preparative TLC eluting with 25% methyl alcohol (containing 2.0 M of ammonia) in chloroform afforded, after isolation of the desired spots (Rf=0.2), 63 mg (25% yield) of N-(6,7-dibutoxy-4-methyl-2-quinazolinyl)guanidine.

Name: 6,7-dibutoxy-2,2,4-trimethyl-1,2-dihydroquinoline. (synthesized using Method B (53% yield)).

Data: ESMS 318 (MH⁺) ; ¹H NMR (CDCl₃) δ6.70 (br s, 1H), 6.07 (br s, 1H), 5.19 (br s, 1H), 3.93 (br s, 4H), 1.94 (br s, 3H), 1.75 (septet, 4H, J=7.8 Hz), 1.48 (septet, 4H, J=7.5 Hz) 1.24 (s, 6H), 0.962 (t, 3H, J=7.2 Hz), 0.958 (t, 3H, J=7.2 Hz).

Compound 1018 (synthesized using Method C (25% yield))

Name: N-(6,7-dibutoxy-4-methyl-2-quinazolinyl)guanidine

Data: ESMS 246 (MH⁺); ¹H NMR (CD₃OD) δ7.89 (br s, 2H), 7.21 (br s, 1H), 7.16 (br s, 1H), 4.13 (t, 2H, J=6.3 Hz), 4.08 (t, 2H, J=6.3 Hz), 2.76 (br s, 3H), 1.88-1.80 (m, 4H), 1.56 (septet, 4H, J=7.5 Hz), 1.013 (t, 3H, J=7.5 Hz), 1.008 (t, 3H, J=7.2 Hz).

### Example 2

The following is a representative example of Methods D-F in Scheme 2 for the synthesis of N-(4-methyl-2-quinazolinyl)guanidine (Compound 1001).

Method D

In a flask equipped with a magnetic stirrer, a solution of 6-bromo-2-fluorobenzoic acid (1.00 g, 4.57 mmol) dissolved in anhydrous ethyl ether (7 mL) was cooled to −78° C. using a dry ice-acetone bath. Methyl lithium was then added dropwise (6.8 mL of a 1.4 M solution in ethyl ether, 9.59 mmol). The reaction was further stirred at −78° C. for 5 min followed by warming to r.t. by removing the dry ice-acetone bath. After stirring for an additional 30 min at r.t., the solution was poured into a mixture of ice and saturated aqueous solution of ammonium chloride. The aqueous phase was extracted with ethyl ether twice and the combined ethereal extracts washed with brine. The organic phase was dried with anhydrous sodium sulfate, filtered and solvent evaporated. Purification by silica column chromatography eluting with 5% ethyl acetate in hexane (Rf=0.4) afforded 194 mg (20% yield) of 1-(5-bromo-2-fluorophenyl)ethanone.

Method E

In a flask equipped with a magnetic stirrer, 1-(5-bromo-2-fluorophenyl)ethanone (517 mg, 2.36 mmol) was dissolved in 1-methyl-2-pyrrolidinone (NMP) (3.4 mL). Dicyandiamide (2.0 eq, 397 mg, 4.72 mmol) and potassium carbonate (1.0 eq, 326 mg, 2.36 mmol) were added to the solution and the reaction was heated at 120° C. for 4 h. Upon cooling the reaction to r.t., the solution was filtered and the residue extracted further with methyl alcohol. The methyl alcohol was evaporated. The NMP solution was placed directly on a silica column eluting with 20% methyl alcohol (containing 2.0 M ammonia) in chloroform. Fractions containing the product (Rf=0.5 with 5% methyl alcohol in ethyl acetate) were combined and solvent evaporated to afford 109 mg (18% yield) of 6-bromo-4-methyl-2-quinazolinylcyanamide.

Method F

To a suspension of ammonium chloride (53.5 mg, 1 mmol) in toluene (1 mL) at r.t. was added 0.5 mL of a 2.0 M trimethylaluminum chloride suspended in toluene (1 mmol). The resulting suspension was stirred at r.t. for 2 h followed by the addition of 4-methyl-2-quinazolinylcyanamide (30 mg, 0.16 mmol). The mixture was heated at 80° C. for 6 h. The reaction mixture was cooled and then poured into a slurry of silica gel in chloroform. The suspension was stirred for 5 min and then filtered. The residue was further washed with methyl alcohol. Purification by preparative TLC eluting with 20% methyl alcohol (containing 2.0 M ammonia) in chloroform (Rf=0.1) afforded N-(4-methyl-2-quinazolinyl)guanidine (11 mg, 34% yield) after isolation of the product.

Compound 1001

Data: ESMS 202 (MH⁺); ¹H NMR (CD₃OD) δ8.15 (d, J=8.1, Hz, 1H), 7.80-7.90 (m, 2H), 7.52-7.58 (m, 1H), 2.89 (s, 3H).

### Example 3

The following is a representative example of Methods G-J in Scheme 3 for the synthesis of N-(6-ethyl-4-methyl-2-quinolinyl)guanidine (Compound 4002).

Method G

To a flask equipped with a magnetic stirrer was added 4-ethylaniline (9.75 g, 80.5 mmol), toluene (20 mL) and methyl acetoacetate (9.1 mL, 85.4 mmol). The reaction mixture was heated to reflux using an Dean-Stark apparatus for 1 h, when the amount of methyl alcohol collected in the apparatus ceased to increase. Upon cooling to r.t., the solvent was evaporated using rotary-evaporator. The crude material was purified by silica column chromatography eluting with 10% methyl alcohol (containing 2.0 M ammonia) in chloroform (Rf=0.6) to afford 5.1 g of N-(4-ethylphenyl)-3-oxobutanamide (31% yield).

Method H

A flask equipped with a magnetic stirrer containing concentrated sulfuric acid (50 mL) was cooled to 0° C. with an ice-bath followed by the addition of water (25 mL). The solution was heated to 80° C. and N-(4-ethylphenyl)-3-oxobutanamide (5.1 g, 24.8 mmol) added. This solution was stirred and heated at 120° C. for 0.5 h. The reaction was then cooled to r.t. and added to a flask containing ice and water (323 mL). Upon standing overnight in water, crystals formed and were collected via filtration. The crystals were dissolved in a minimum amount of methyl alcohol and filtered through a short pad of silica eluting with 10% methyl alcohol (containing 2.0 M of ammonia) in chloroform. Evaporation of the solvent afforded 3.06 g (66% yield) of 6-ethyl-4-methyl-2(1H)-quinolinone.

Method I

To a flask equipped with a magnetic stirrer were added 6-ethyl-4-methyl-2(1H)-quinolinone (3.06 g, 16.3 mmol) and phosphorus oxychloride (16.3 mL, 16.3 mmol). The mixture was refluxed for 18 h.) The solution was cooled to r.t. and poured into ice water (163 mL) and neutralized to pH=7 using 6 N NaOH (aq). The aqueous phase was extracted with methylene chloride (3×). The organic phase was then filtered through a short pad of silica eluting with methylene chloride. Evaporation of the solvent afforded 2.60 g (77% yield) of 2-chloro-6-ethyl-4-methylquinoline.

Method J

To a flask equipped with a magnetic stirrer were added 2-chloro-6-ethyl-4-methylquinoline (2.02 g, 9.81 mmol), 1-methyl-2-pyrrolidinone (41 mL), potassium carbonate (3.12 g, 22.6 mmol) and guanidine hydrochloride (1.12 g, 11.8 mmol). The mixture was heated at 140° C. for 12 h. Upon cooling to r.t., the mixture was filtered and the residue further extracted with methyl alcohol. The filtrates were combined and the solvent evaporated. The crude material was purified by reverse phase HPLC to afford 46 mg (1% yield) of N-(6-ethyl-4-methyl-2-quinolinyl)guanidine as the trifluoroacetate salt.

Name: N-(4-ethylphenyl)-3-oxobutanamide. (synthesized using Method G (31% yield)).

Data: ESMS 206 (MH⁺); ¹H NMR (CD₃OD) δ7.42 (d, 2H, J=8.4 Hz), 7.13 (d, 2H, J=8.4 Hz), 3.29 (s, 2H), 2.59 (q, 2H, J=7.8 Hz), 2.25 (s, 3H), 1.19 (t, 3H, J=7.5 Hz).

Name: 6-ethyl-4-methyl-2(1H)-quinolinone. (synthesized using Method H (66% yield)).

Data: ESMS 188 (MH⁺); ¹H NMR (CDCl₃) δ7.55 (s, 1H), 7.50 (d, 1H, J=8.4 Hz), 7.47 (d, 1H, J=8.4 Hz), 6.69 (s, 1H), 2.77 (q, 2H, J=7.8 Hz), 2.59 (s, 3H), 1.30 (t, 3H, J=7.8 Hz).

Name: 2-chloro-6-ethyl-4-methylquinoline (synthesized using Method I (77% yield)).

Data: ESMS 208 & 206 (MH⁺); ¹H NMR (CD₃OD) δ7.80 (br d, 1H, J=8.7 Hz), 7.63 (dd, 1H, J=8.7, 1.8 Hz), 7.29 (d, 1H, J=0.6 Hz), 2.84 (q, 2H, J=7.5 Hz), 2.66 (d, 3H, J=0.9 Hz), 1.31 (t, 3H, J=7.5 Hz).

Compound 4002 (class: Quinolino-guanidine; synthesized using Method J).

Name: N-(6-ethyl-4-methyl-2-quinolinyl)guanidine.

Data: ESMS 229 (MH⁺) ; ¹H NMR (CD₃OD) δ7.77 (br d, 1H, J=8.7 Hz), 7.57 (dd, 1H, J=8.7, 1.8 Hz), 6.90 (d, 1H, J=0.6 Hz), 2.81 (q, 2H, J=7.5 Hz), 2.64 (d, 3H, J=0.6 Hz), 1.30 (t, 3H, J=7.5 Hz).

### Example 4

Compound 3001 (Purchased from Tripos (St. Lousis, Mo.)).

Name: N-(4,7-dimethyl-2-quinazolinyl)guanidine.

### Example 5

Compound 1007 (class: Quinazolino-guanidine; Purchased from Sigma)

Name: N-(1-methylbenzo[f]quinazolin-3-yl)guanidine.

### Example 6

N-(4-methyl-2-quinolinyl)guanidine is made in the same manner as N-(6-ethyl-4-methyl-2-quinolinyl)guanidine (see Example 3) except that 2-chloro-4-methylquinoline is used in place of 2-chloro-6-ethyl-4-methylquinoline.

Compound 6001 (class: Quinolino-guanidine; synthesized using Method J (67% yield))

Name: N-(4-methyl-2-quinolinyl)guanidine.

Data: ESMS 201 (MH⁺) ; ¹H NMR (CD₃OD) δ7.86 (d, J=8.1 Hz, 1H), 7.70 (d, J=8.4 Hz, 1H), 7.52-7.59 (m, 1H), 7.32-7.38 (m, 1H), 6.80 (s, 1H), 2.57 (s, 3H) ; Anal. (C₁₁H₁₂N₄. 0.15 CHCl₃) calcd, C, 61.39; H, 5.61; N, 25.68; Found, C, 61.81; H, 5.40; N, 26.36.

### Example 7

N-(4,7-dimethyl-2-quinolinyl)guanidine is made in the same manner as N-(6-ethyl-4-methyl-2-quinolinyl)guanidine (see Example 3) except that 3-methylaniline is used in place of 4-ethylaniline.

Compound 4006 (Class: Quinolino-guanidine; synthesized using Method J (17% yield))

Name: N-(4,7-dimethyl-2-quinolinyl)guanidine.

Data: ESMS 215 (MH⁺); ¹H NMR (CD₃OD) δ7.89 (d, J=8.5 Hz, 1H), 7.67 (s, 1H), 7.37 (dd, J=8.5, 1.6 Hz, 1H), 6.88 (s, 1H), 2.65 (s, 3H), 2.51 (s, 3H).

### Example 8

N-(4-ethyl-7-methyl-2-quinolinyl)guanidine is made in the same manner as N-(6-ethyl-4-methyl-2-quinolinyl)guanidine (see Example 3) except that 3-methylaniline is used in place of 4-ethylaniline and methyl-3-oxopentanoate in place of methyl acetoacetate.

Compound 6003 (class: Quinolino-guanidine; synthesized using Method J (9% yield))

Name: N-(4-ethyl-7-methyl-2-quinolinyl)guanidine.

Data: ESMS 229 (MH⁺); ¹H NMR (CD₃OD) δ7.92 (d, J=8.6 Hz, 1H), 7.68 (s, 1H), 7.37 (dd, J=8.5, 1.7 Hz, 1H), 6.90 (s, 1H), 3.07 (q, J=7.2 Hz, 2H), 2.51 (s, 3H), 1.36 (t, J=7.5 Hz, 3H).

### Example 9

N-(4,8-dimethyl-2-quinolinyl)guanidine is made in the same manner as N-(6-ethyl-4-methyl-2-quinolinyl)guanidine (see Example 3) except that 2-chloro-4,8-dimethylquinoline is used in place of 2-chloro-6-ethyl-4-methylquinoline.

Compound 6002 (class: Quinolino-guanidine; synthesized using Method J (20% yield))

Name: N-(4,8-dimethyl-2-quinolinyl)guanidine.

Data: ESMS 215 (MH⁺); ¹H NMR (CD₃OD) δ7.84 (d, J=8.1 Hz, 1H), 7.57 (d, J=7.2 Hz, 1H), 7.41 (dd, J=8.1, 7.2 Hz, 1H), 6.94 (d, J=0.6 Hz, 1H), 2.66 (s, 3H), 2.56 (s, 3H).

### Example 10

N-(6-chloro-4-methyl-2-quinolinyl)guanidine is made in the same manner as N-(6-ethyl-4-methyl-2-quinolinyl)guanidine (see Example 3) except that 2,6-dichloro-4-methylquinoline is used in place of 2-chloro-6-ethyl-4-methylquinoline.

Compound 4005 (class: Quinolino-guanidine; synthesized using Method J (42-71% yield)).

Name: N-(6-chloro-4-methyl-2-quinolinyl)guanidine.

Data: ESMS 231 (MH⁺); ¹H NMR (CD₃OD) δ7.80 (d, J=2.4 Hz, 1H), 7.88 (d, J=8.7 Hz, 1H), 7.66 (dd, J=9.0, 2.4 Hz, 1H), 7.00 (d, J=0.9 Hz, 1H), 2.65 (s, 3H) ; Anal. (C₁₁H₁₁ClN₄+0.1 CHCl₃. 0.7 H₂O) calcd, C, 51.43; H, 4.86; N, 21.61; Found, C, 51.41; H, 4.85; N, 21.78.

### Example 11

N-(1-methylbenzo[f]quinolin-3-yl) guanidine is made in the same manner as N-(6-ethyl-4-methyl-2-quinolinyl)guanidine (see Example 3) except that 3-chloro-1-methylbenzo[f]quinoline is used in place of 2-chloro-6-ethyl-4-methylquinoline.

Compound 4009 (class: Quinolino-guanidine; synthesized using Method J (21% yield))

Name: N-(1-methylbenzo[f]quinolin-3-yl)guanidine.

Data: ESMS 251 (MH⁺); ¹H NMR (CD₃OD) δ8.63 (d, J=7.8 Hz, 1H), 7.83-7.87 (m, 2H), 7.46-7.63 (m, 3H), 6.91 (s, 1H), 2.93 (s, 3H).

### Example 12

N-(6-methoxy-4-methyl-2-quinolinyl)guanidine is made in the same manner as N-(6-ethyl-4-methyl-2-quinolinyl)guanidine (see Example 3) except that 2-chloro-6-methoxy-4-methylquinoline is used in place of 2-chloro-6-ethyl-4-methylquinoline.

Compound 4004 (class: Quinolino-guanidine; synthesized using Method J (13% yield)).

Name: N-(6-methoxy-4-methyl-2-quinolinyl)guanidine.

Data: ESMS 231 (MH⁺); ¹H NMR (CD₃OD) δ7.80 (d, J=9.3 Hz, 1H), 7.34 (dd, J=9.0, 2.7 Hz, 1H), 6.98 (d, J=0.9 Hz, 1H), 3.92 (s, 3H), 2.65 (s, 3H).

### Example 13

N-(4,5,7-trimethyl-2-quinolinyl)guanidine is made in the same manner as N-(6-ethyl-4-methyl-2-quinolinyl)guanidine (see Example 3) except that 3,5-dimethylaniline is used in place of 4-ethylaniline.

Compound 4008 (class: Quinolino-guanidine; synthesized using Method J (7% yield)).

Name: N-(4,5,7-trimethyl-2-quinolinyl)guanidine.

Data: ESMS 229 (MH⁺); ¹H NMR (CD₃OD) δ7.51 (s, 1H), 7.13 (s, 1H), 6.80 (s, 1H), 2.85 (s, 3H), 2.82 (s, 3H), 2.42 (s, 3H).

### Example 14

N-(4,6-dimethyl-2-quinolinyl)guanidine is made in the same manner as N-(6-ethyl-4-methyl-2-quinolinyl)guanidine (see Example 3) except that 4-methylaniline is used in place of 4-ethylaniline.

Compound 4001 (class: Quinolino-quanidine; synthesized using Method J (5% yield)).

Name: N-(4,6-dimethyl-2-quinolinyl)guanidine.

Data: ESMS 215 (MH⁺); ¹H NMR (CD₃OD) δ7.79 (dd, J=4.2, 4,2 Hz, 2H), 7.89 (dd, J=8.7, 1.8 Hz, 1H), 7.75 (d, J=0.9 Hz, 1H), 2.67 (d, J=0.9 Hz, 3H), 2.52 (s, 3H).

### Example 15

N-(4-methyl-6-phenyl-2-quinolinyl)guanidine is made in the same manner as N-(6-ethyl-4-methyl-2-quinolinyl)guanidine (see Example 3) except that 2-chloro-4-methyl-6-phenylquinoline is used in place of 2-chloro-6-ethyl-4-methylquinoline.

Compound 4003 (class: Quinolino-guanidine; synthesized using Method J (28% yield)).

Name: N-(4-methyl-6-phenyl-2-quinolinyl)guanidine.

Data: ESMS 277 (MH⁺); ¹H NMR (CD₃OD) δ8.10 (d, J=1.2 Hz, 1H), 7.90-7.98 (m, 2H), 7.65-7.73 (m, 2H), 7.32-7.50 (m, 3H) 7.01 (s, 1H), 2.73 (s, 3H).

### Example 16

N-(7-ethyl-4-methyl-2-quinazolinyl)guanidine is made in the same manner as N-(6-ethyl-4-methyl-2-quinolinyl)guanidine (see Example 3) except that 3-ethylaniline is used in place of 4-ethylaniline.

Compound 1020 (class: Quinazolino-guanidine; synthesized using Method C (52% yield)).

Name: N-(7-ethyl-4-methyl-2-quinazolinyl)guanidine.

Data: ESMS 230 (MH⁺); ¹H NMR (CD₃OD) δ8.09 (d, J=8.4 Hz, 1H), 7.68 (d, J=0.9 Hz, 1H), 7.49 (dd, J=8.4, 1.5 Hz, 1H), 2.88 (s, 3H), 2.86 (q, J=7.6 Hz, 2H), 1.32 (t, J=7.5 Hz, 3H).

### Example 17

N-(7-fluoro-4-methyl-2-quinolinyl)guanidine is made in the same manner as N-(6-ethyl-4-methyl-2-quinolinyl)guanidine (see Example 3) except that 3-fluoroaniline is used in place of 4-ethylaniline.

Compound 4007 (class: Quinolino-quanidine; synthesized using Method J (36% yield)).

Name: N-(7-fluoro-4-methyl-2-quinolinyl)guanidine.

Data: ESMS 219 (MH⁺); ¹H NMR (CD₃OD) δ8.00 (dd, J=9.0, 6.0 Hz, 1H), 7.57 (dd, J=10.2, 2.4 Hz, 1H), 7.30 (dt, J=8.7, 2.7 Hz, 1H), 6.88 (s, 1H), 2.64 (s, 3H); Anal. (C₁₁H₁₁FN₄ 1.1 CF₃CO₂H) calcd, C, 46.13; H, 3.55; N, 16.30; Found, C, 46.66; H, 3.31; N, 16.41.

### Example 18

Compound 1002 (class: Quinazolino-quanidine).

Name: N-(4,6-dimethyl-2-quinazolinyl)guanidine.

A compound purchased from Tripos was found to have the wrong structure assignment and to contain an impurity. Tripos' incorrect structure assignment was 2-[(4,7-dimethyl-2-quinazolinyl)amino]-4-quinazolinol. By NMR and MS techniques, the sample was determined to be a mixture of N-(4,6-dimethyl-2-quinazolinyl)guanidine and methyl 2-aminobenzoate, which was separated by preparative TLC to afford pure N-(4,6-dimethyl-2-quinazolinyl)guanidine.

Data: ESMS 216 (MH⁺—NH₃); ¹H NMR (CD₃OD) δ7.97 (s, 1H), 7.77 (br s, 2H, 2^{nd} Order Coupling), 2.89 (s, 3H), 2.54 (s, 3H); ¹³C NMR (CD₃OD) 172.2, 156.4, 153.4, 147.8, 137.7, 137.6, 127.0, 124.9, 122.1, 21.0, 20.7.

### Example 19

N-(6,7-difluoro-4-methyl-2-quinazolinyl)guanidine is made in the same manner as N-(6,7-dibutoxy-4-methyl-2-quinazolinyl)guanidine (see Example 1, steps B and C) except that 3,4-difluoroaniline is used in place of 3,4-dibutoxyaniline.

Compound 1019 (class: Quinolino-guanidine; synthesized using Method J (42% yield)).

Name: N-(6,7-difluoro-4-methyl-2-quinazolinyl)guanidine.

Data: ESMS 238 (MH⁺); ¹H NMR (CD₃OD) δ7.98 (dd, J=10.8, 8.7 Hz, 1H), 7.59 (dd, J=11.4, 7.5 Hz, 1H), 2.80 (s, 3H); Anal. (C₁₀H₉F₂N₅. 0.21 SiO₂) calcd, C, 48.08; H, 3.63; N, 28.03; Found, C, 47.61; H, 3.61; N, 28.46.

### Example 20

N-(7-bromo-4-methyl-2-quinazolinyl)guanidine is made in the same manner as N-(6,7-dibutoxy-4-methyl-2-quinazolinyl)guanidine (see Example 1) except that 3-bromoaniline is used in place of 3,4-dibutoxyaniline.

Name: 7-bromo-2,2,4-trimethyl-1,2-dihydroquinoline (Synthesized using Method B (28%)).

Data: ESMS 254 & 252 (MH⁺); ¹H NMR (CDCl₃) δ6.88 (d, 1H, J=8.1 Hz), 6.72 (dd, 1H, J=8.1, 2.1 Hz), 6.57 (d, 1H, J=2.1 Hz), 5.31 (br d, 1H, J=1.2 Hz), 1.95 (d, 3H, J=1.5 Hz), 1.27 (s, 6H).

Compound 1014 (class: Quinazolino-quanidine; synthesized using Method C (7% yield)).

Name: N-(7-bromo-4-methyl-2-quinazolinyl)guanidine.

Data: ESMS 282 & 280 (MH⁺); ¹H NMR (CD₃OD) δ8.08 (d, 1H, 7.8 Hz), 7.88 (s, 1H), 7.69 (br d, 1H, J=8.7 Hz), 2.89 (s, 3H).

### Example 21

N-(6-bromo-4-methyl-2-quinazolinyl)guanidine is made in the same manner as N-(6,7-dibutoxy-4-methyl-2-quinazolinyl)guanidine (see Example 1) except that 4-bromoaniline is used in place of 3,4-dibutoxyaniline.

Name: 6-bromo-2,2,4-trimethyl-1,2-dihydroquinoline. (Synthesized using Method B (22% yield)).

Data: ESMS 254 & 252 (MH⁺); ¹H NMR (CDCl₃) δ7.12 (d, 1H, J=2.1 Hz), 7.04 (dd, 1H, J=8.4, 2.1 Hz), 6.31 (br d, 1H, J=8.4 Hz), 5.33 (br s, 1H), 1.95 (d, 3H, J=1.5 Hz), 1.26 (s, 6H).

Compound 1026 (class: Quinazolino-guanidine; synthesized using Methods C (4% yield)).

Name: N-(6-bromo-4-methyl-2-quinazolinyl)guanidine.

Data: ESMS 282 & 280 (MH⁺); ¹H NMR (CD₃OD) δ8.40 (d, 1H, J=2.1 Hz), 8.02 (dd, 1H, J=8.7, 2.1 Hz), 7.85 (d, 1H, J=9.0 Hz), 2.91 (s, 3H).

### Example 22

N-[4-methyl-7-(trifluoromethoxy)-2-quinazolinyl]guanidine is made in the same manner as N-(6,7-dibutoxy-4-methyl-2-quinazolinyl)guanidine (see Example 1) except that 3-trifluoromethoxyaniline is used in place of 3,4-dibutoxyaniline.

Name: 2,2,4-trimethyl-7-(trifluoromethoxy)-1,2-dihydroquinoline (Synthesized using Method B (29% yield)).

Data: ESMS 258 (MH⁺); ¹H NMR (CDCl₃) δ7.00 (d, 1H, J=8.1 Hz), 6.44 (dd, 1H, J=7.5, 1.2 Hz), 6.26 (br s, 1H), 5.30 (d, 1H,J=1.5 Hz), 1.96 (d, 3H, J=1.5 Hz), 1.28 (s, 6H).

Compound 1036

Name: N-[4-methyl-7-(trifluoromethoxy)-2-quinazolinyl]guanidine (class: Quinazolino-quanidine; synthesized using Method C (5% yield).

Data: ESMS 286 (MH⁺); ¹H NMR (CD₃OD) δ8.26 (d, 1H, J=9.3 Hz), 7.69 (br s, 1H), 7.39 (dm, 1H, J=7.2 Hz), 2.89 (s, 3H).

### Example 23

N-(6-chloro-4-methyl-2-quinazolinyl)guanidine is made in the same manner as N-(6,7-dibutoxy-4-methyl-2-quinazolinyl)guanidine (see Example 1) except that 4-chloroaniline is used in place of 3,4-dibutoxyaniline.

Compound 1013

Name: N-(6-chloro-4-methyl-2-quinazolinyl)guanidine (class: Quinazolino-guanidine; synthesized using Method C (35% yield)).

Data: ESMS 236 (MH⁺); ¹H NMR (CD₃OD) δ8.20 (t, J=1.5 Hz, 1H), 7.86 (d, J=1.5 Hz, 2H), 2.89 (s, 3H); Anal. (C₁₀H₁₀ClN₅. 0.21 CHCl₃. 0.7 H₂O) calcd, C, 44.86; H, 4.28; N, 25.62; Found, C, 44.62; H, 4.28; N, 25.91.

### Example 24

N-(6-methoxy-4-methyl-2-quinazolinyl)guanidine is made in the same manner as N-(6,7-dibutoxy-4-methyl-2-quinazolinyl)guanidine (see Example 1) except that 4-methoxyaniline is used in place of 3,4-dibutoxyaniline.

Compound 1011 (class: Quinazolino-quanidine; synthesized using Method C (13% yield)).

Name: N-(6-methoxy-4-methyl-2-quinazolinyl)guanidine.

Data: ESMS 232 (MH⁺); ¹H NMR (CD₃OD) δ7.77 (d, J=9.0 Hz, 1H), 7.54 (dd, J=9.3, 2.7 Hz, 1H), 7.38 (d, J=2.7 Hz, 1H), 3.94 (s, 3H), 2.87 (s, 3H).

### Example 25

N-(7-isopropyl-4-methyl-2-quinazolinyl)guanidine is made in the same manner as N-(6,7-dibutoxy-4-methyl-2-quinazolinyl)guanidine (see Example 1) except that 3-isopropylaniline is used in place of 3,4-dibutoxyaniline.

Compound 1021 (class: Quinazolino-quanidine; synthesized using Method C (85%), except that reverse phase (C18) column chromatography eluting with acetonitrile was used in place of normal phase).

Name: N-(7-isopropyl-4-methyl-2-quinazolinyl)guanidine.

Data: ESMS 244 (MH⁺); ¹H NMR (CD₃OD) δ8.11 (d, 1H, J=8.4 Hz), 7.72 (d, 1H, J=1.5 Hz), 7.54 (dd, 1H, J=8.7, 1.8 Hz), 3.12 (septet, 1H, J=6.9 Hz), 2.88 (s, 3H), 1.34 (d, 6H, J=6.9 Hz).

### Example 26

N-[4-methyl-6-(trifluoromethoxy)-2-quinazolinyl]guanidine is made in the same manner as N-(6,7-dibutoxy-4-methyl-2-quinazolinyl)guanidine (see Example 1) except that 4-trifluoromethoxyaniline is used in place of 3,4-dibutoxyaniline.

Name: 2,2,4-trimethyl-6-(trifluoromethoxy)-1,2-dihydroquinoline. (Synthesized using Method B (19% yield)).

Data: ESMS 258 (MH⁺); ¹H NMR (CDCl₃) δ6.89 (br d, 1H, J=1.8 Hz), 6.83 (br dd, 1H, J=8.7, 1.5 Hz), 6.37 (d, 1H, J=8.4 Hz), 5.37 (br s, 1H), 1.96 (d, 3H, J=1.2 Hz), 1.28 (s, 6H).

Compound 1030 (synthesized using Method C (11% yield)).

Name: N-[4-methyl-6-(trifluoromethoxy)-2-quinazolinyl]guanidine.

Data: ESMS 286 (MH⁺); ¹H NMR (CD₃OD) δ8.02 (br d, 1H, J=2.1 Hz), 7.90 (d, 1H, J=9.3 Hz), 7.77 (br dd, 1H, J=8.7, 1.8 Hz), 2.88 (s, 3H).

### Example 27

N-(4-methyl-6-pentyl-2-quinazolinyl)guanidine is made in the same manner as N-(6,7-dibutoxy-4-methyl-2-quinazolinyl)guanidine (see Example 1) except that 4-pentylaniline is used in place of 3,4-dibutoxyaniline.

Name: 2,2,4-trimethyl-6-pentyl-1,2-dihydroquinoline (synthesized using Method B (32% yield).

Data: ESMS 244 (MH⁺); ¹H NMR (CDCl₃) δ6.86 (d, 1H, J=0.9 Hz), 6.80 (dd, 1H, J=7.8, 0.9 Hz), 6.37 (d, 1H, J=7.8 Hz), 5.30 (br s, 1H), 2.47 (t, 2H, J=7.5 Hz), 1.98 (d, 3H, J=0.9 Hz), 1.54 (br p, 2H, J=7.2 Hz), 1.34-1.25 (m, 4H), 1.26 (s, 6H), 0.88 (br t, 3H, J=6.6 Hz).

Compound 2001

Name: N-(4-methyl-6-pentyl-2-quinazolinyl)guanidine (synthesized using Method C (9-41% yield). crystallization from MeOH and reverse phase (C18) HPLC were required).

Data: ESMS 272 (MH⁺); ¹H NMR (CD₃OD) δ7.97 (s, 1H, 2^{nd} order coupling), 7.81 (br s, 2H, 2^{nd} order coupling), 2.91 (s, 3H), 2.82 (t, 2H, J=7.8 Hz), 1.73-1.68 (m, 2H), 1.38-1.34 (m, 4H), 0.90 (br t, 3H, J=6.6 Hz).

### Example 28

N-(4,6,7-trimethyl-2-quinazolinyl)guanidine is made in the same manner as N-(6,7-dibutoxy-4-methyl-2-quinazolinyl)guanidine (see Example 1) except that 3,4-dimethylaniline is used in place of 3,4-dibutoxyaniline.

Name: 2,2,4,6,7-pentamethyl-1,2-dihydroquinoline (synthesized using Method B (47% yield)).

Data: ¹H NMR (CDCl₃) δ6.82 (s, 1H), 6.28 (s, 1H), 5.24 (d, 1H, J=0.9 Hz), 2.14 (s, 6H), 1.96 (d, 3H, J=1.2 Hz), 1.24 (s, 6H).

Compound 1015 (class: Quinazolino-guanidine; synthesized using Method C (12% yield)).

Name: N-(4,6,7-trimethyl-2-quinazolinyl)guanidine.

Data: ESMS 230 (MH⁺); ¹H NMR (CD₃OD) δ7.93 (s, 1H), 7.66 (s, 1H), 2.87 (s, 3H), 2.48 (s, 3H), 2.47 (s, 3H).

### Example 29

N-[6-(benzyloxy)-4-methyl-2-quinazolinyl]guanidine is made in the same manner as N-(6,7-dibutoxy-4-methyl-2-quinazolinyl)guanidine (see Example 1) except that 4-benzyloxyaniline is used in place of 3,4-dibutoxyaniline.

Name: 6-(benzyloxy)-2,2,4-trimethyl-1,2-dihydroquinoline (synthesized using Method B (60% yield)).

Data: ESMS 280 (MH⁺).

Compound 1028 (class: Quinazolino-guanidine; synthesized using Method C (6% yield)).

Name: N-[6-(benzyloxy)-4-methyl-2-quinazolinyl]guanidine.

Data: ESMS 308 (MH⁺); ¹H NMR (CD₃OD) δ7.83 (br d, 1H, J=9.0 Hz), 7.66 (br d, 1H, J=9.0 Hz), 7.55-7.48 (m, 3H), 7.40-4.31 (m, 4H), 5.25 (s, 2H), 2.87 (s, 3H).

### Example 30

N-[7-(1-hydroxyethyl)-4-methyl-2-quinazolinyl]guanidine is made in the same manner as N-(6,7-dibutoxy-4-methyl-2-quinazolinyl)guanidine (see Example 1) except that 3-(1-hydroxyethyl)aniline is used in place of 3,4-dibutoxyaniline.

Compound 1035

Name: N-[7-(1-hydroxyethyl)-4-methyl-2-quinazolinyl]guanidine (synthesized using Method C (86% yield)).

Data: ESMS 246 (MH⁺); ¹H NMR (CD₃OD) δ8.17 (d, 1H, J=8.7 Hz), 7.87 (s, 1H), 7.64 (d, 1H, J=8.7 Hz), 5.02 (q, 1H, J=6.6 Hz), 2.91 (br s, 3H), 1.50 (d, 3H, J=6.6 Hz).

### Example 31

N-(6-ethyl-4-methyl-2-quinazolinyl)guanidine is made in the same manner as N-(6,7-dibutoxy-4-methyl-2-quinazolinyl)guanidine (see Example 1) except that 4-ethylaniline is used in place of 3,4-dibutoxyaniline.

Name: 6-ethyl-2,2,4-trimethyl-1,2-dihydroquinoline (synthesized using Method B (38% yield)).

Data: ESMS 202 (MH⁺); ¹H NMR (CDCl₃) δ6.89 (d, 1H, J=1.5 Hz), 6.83 (dd, 1H, J=8.1, 1.8 Hz), 6.39 (d, 1H, J=8.1 Hz), 5.31 (d, 1H, J=0.9 Hz), 2.52 (q, 2H, J=7.5 Hz), 1.99 (d, 3H, J=1.2 Hz), 1.26 (s, 6H), 1.19 (t, 3H, J=7.5 Hz).

Compound 1003 (class: Quinazolino-guanidine; synthesized using Method C (7% yield)).

Name: N-(6-ethyl-4-methyl-2-quinazolinyl)guanidine.

Data: ESMS 230 (MH⁺); ¹H NMR (CD₃OD) δ7.97 (br s, 1H, 2^{nd} order coupling), 7.818 (s, 1H, 2^{nd} order coupling), 7.815 (s, 1H, 2^{nd} order coupling), 2.91 (s, 3H), 2.85 (q, 2H, J=7.5 Hz), 1.32 (t, 3H, J=7.5 Hz).

### Example 32

N-(6-sec-butyl-4-methyl-2-quinazolinyl)guanidine is made in the same manner as N-(6,7-dibutoxy-4-methyl-2quinazolinyl)guanidine (see Example 1) except that 4-sec-butylaniline is used in place of 3,4-dibutoxyaniline.

Name: 6-sec-butyl-2,2,4-trimethyl-1,2-dihydroquinoline (synthesized using Method B (50% yield)).

Data: ESMS 230 (MH⁺); ¹H NMR (CDCl₃) δ6.86 (br s, 1H), 6.80 (br d, 1H, J=8.7 Hz), 6.39 (br d, 1H, J=8.5 Hz), 5.30 (br s, 1H), 2.50-2.40 (m, 1H), 1.99 (s, 3H), 1.53 (q, 2H, J=7.2 Hz), 1.27 (s, 6H), 1.19 (d, 3H, J=6.9 Hz), 0.82 (t, 3H, J=7.5 Hz).

Compound 2002 (class: Quinazolino-guanidine; synthesized using Method C (36% yield)).

Name: N-(6-sec-butyl-4-methyl-2-quinazolinyl)guanidine.

Data: ESMS 258 (MH⁺); ¹H NMR (CD₃OD) δ7.90 (s, 1H, 2^{nd} order coupling), 7.787 (s, 1H, 2^{nd} order coupling), 7.791 (s, 1H, 2^{nd} order coupling), 2.88 (s, 3H), 2.83 (septet, 1H, J=7.2 Hz), 1.69 (p, 2H, J=7.2 Hz), 1.31 (d, 3H, J=6.9 Hz), 0.83 (t, 3H, J=7.2 Hz).

### Example 33

N-(4-methylfuro[2,3-g]quinazolin-2-yl)guanidine is made in the same manner as N-(6,7-dibutoxy-4-methyl-2-quinazolinyl)guanidine (see Example 1) except that 5-nitro-[2,3]-benzofuran is used in place of 1,2-dibutoxy-4-nitrobenzene.

Name: 6,6,8-trimethyl-5,6-dihydrofuro[2,3-g]quinoline (synthesized using Method B (70% yield)).

Data: ¹H NMR (CDCl₃) δ7.53 (br s, 1H), 7.21 (dd, 1H, J=8.4, 0.6 Hz), 6.94 (br s, 1H), 6.51 (d, 1H, J=8.4 Hz), 5.38 (d, 1H, J=1.2 Hz), 2.29 (d, 3H, J=1.2 Hz), 1.29 (s, 6H).

Compound 1039

Name: N-(4-methylfuro[2,3-g]quinazolin-2-yl)guanidine (class: Quinazolino-guanidine; synthesized using Method C (85% yield)).

Data: ESMS 242 (MH⁺); ¹H NMR (CD₃OD) δ8.18 (d, 1H, J=9.6 Hz), 8.14 (br s, 1H,), 7.85 (d, 1H, J=9.0 Hz), 7.53 (br s, 1H), 3.13 (s, 3H).

### Example 34

N-(6-butoxy-4-methyl-2-quinazolinyl)guanidine is made in the same manner as N-(6,7-dibutoxy-4-methyl-2-quinazolinyl)guanidine (see Example 1) except that 4-butoxyaniline is used in place of 3,4-dibutoxyaniline.

Name: butyl 2,2,4-trimethyl-1,2-dihydro-6-quinolinyl ether. (synthesized using Method B (14% yield)).

Data: ESMS 246 (MH⁺); ¹H NMR (CDCl₃) δ6.69 (br d, 1H, J=2.7 Hz), 6.60 (dd, 1H, J=8.4, 2.7 Hz), 6.40 (d, 1H, J=8.4 Hz), 5.36 (br s, 1H), 3.89 (t, 2H, J=6.6 Hz), 1.97 (d, 3H, J=0.9 Hz), 1.72 (p, 2H, J=5.7 Hz), 1.47 (septet, 2H, J=7.2 Hz), 1.25 (s, 6H), 0.96 (t, 3H, J=7.2 Hz).

Compound 1012 (class: Quinazolino-guanidine; synthesized using Method C (12% yield)).

Name: N-(6-butoxy-4-methyl-2-quinazolinyl)guanidine.

Data: ESMS 247 (MH); ¹H NMR (CD₃OD) δ7.81 (d, 1H, J=9.0 Hz), 7.56 (dm, 1H, J=9.3 Hz), 7.50-7.40 (m, 1H), 4.14 (t, 2H, J=6.0 Hz), 2.89 (s, 3H), 1.84 (p, 2H, J=7.8 Hz), 1.55 (septet, 2H, J=7.5 Hz), 1.01 (t, 3H, J=7.5 Hz).

### Example 35

N-(4-methyl-6-phenoxy-2-quinazolinyl)guanidine is made in the same manner as N-(6,7-dibutoxy-4-methyl-2-quinazolinyl)guanidine (see Example 1) except that 4-phenoxyaniline is used in place of 3,4-dibutoxyaniline.

Name: 2,2,4-trimethyl-6-phenoxy-1,2-dihydroquinoline (synthesized using Method B (10% yield).

Data: ¹H NMR (CDCl₃) δ7.187 (t, 2H, J=7.8 Hz), 6.91 (t, 1H, J=6.9 Hz), 6.81 (d, 2H, J=7.8 Hz), 6.68 (d, 1H, J=2.1 Hz), 6.60 (dd, 1H, J=8.4, 2.1 Hz), 6.53 (d, 1H, J=8.4 Hz), 5.37 (br s, 1H), 1.88 (d, 3H, J=1.2 Hz), 1.23 (s, 6H).

Compound 1032 (class: Quinazolino-guanidine; synthesized using Method C (11% yield)).

Name: N-(4-methyl-6-phenoxy-2-quinazolinyl)guanidine.

Data: ESMS 294 (MH⁺); ¹H NMR (CD₃OD) δ7.93 (d, 1H, J=9.0 Hz), 7.66 (dd, 1H, J=9.0, 2.7 Hz), 7.58 (d, 1H, J=2.7 Hz), 7.42 (t, 2H, J=7.5 Hz), 7.20 (t, 1H, J 7.5 Hz), 7.09 (br d, 2H, J=7.5 Hz), 2.79 (s, 3H).

### Example 36

N-(6-cyclohexyl-4-methyl-2-quinazolinyl)guanidine is made in the same manner as N-(6,7-dibutoxy-4-methyl-2-quinazolinyl)guanidine (see Example 1) except that 4-cyclohexylaniline is used in place of 3,4-dibutoxyaniline.

Name: 6-cyclohexyl-2,2,4-trimethyl-1,2-dihydroquinoline. (synthesized using Method B (47% yield).

Data: ¹H NMR (CDCl₃) δ7.00 (d, 1H, J=1.8 Hz), 6.94 (dd, 1H, J=8.1, 1.8 Hz), 6.45 (3, 1H, J=8.1 Hz), 5.38 (d, 1H, J=1.2 Hz), 2.55-2.42 (m 1H), 2.09 (s, 3H), 1.97-1.91 (m, 5H), 1.83 (br d, 1H, J=12 Hz), 1.55-1.42 (m, 4H), 1.34 (s, 6H).

Compound 1029 (class: Quinazolino-guanidine; synthesized using Method C (14% yield)).

Name: N-(6-cyclohexyl-4-methyl-2-quinazolinyl)guanidine.

Data: ESMS 284 (MH⁺).

### Example 37

N-[4-methyl-6-(pentyloxy)-2-quinazolinyl]guanidine is made in the same manner as N-(6,7-dibutoxy-4-methyl-2-quinazolinyl)guanidine (see Example 1) except that 4-pentyloxyaniline is used in place of 3,4-dibutoxyaniline.

Name: Pentyl 2,2,4-trimethyl-1,2-dihydro-6-quinolinyl ether. (synthesized using Method B (59% yield)

Data: ESMS 260 (MH⁺).

Compound 1031 (class: Quinazolino-guanidine; synthesized using Method C (13% yield)).

Name: N-[4-methyl-6-(pentyloxy)-2-quinazolinyl]guanidine.

Data: ESMS 288 (MH⁺); ¹H NMR (CD₃OD) δ7.82 (d, 1H, J=9.3 Hz), 7.57 (dd, 1H, J=9.0, 2.4 Hz), 7.41 (d, 1H, J=2.7 Hz), 4.13 (t, 2H, J=6.3 Hz), 2.89 (s, 3H), 1.86 (br p, 2H, J=7.2 Hz), 1.55-1.35 (m, 4H), 0.95 (br t, 3H, J=7.2 Hz).

### Example 38

N-[4-methyl-6-(4-methylphenoxy)-2-quinazolinyl]guanidine is made in the same manner as N-(6,7-dibutoxy-4-methyl-2-quinazolinyl)guanidine (see Example 1) except that 4-(4-methylphenoxy)aniline is used in place of 3,4-dibutoxyaniline.

Name: 2,2,4-trimethyl-6-(4-methylphenoxy)-1,2-dihydroquinoline (synthesized using Method B (27% yield)).

Data: ESMS 280 (MH⁺).

Compound 1033 (class: Quinazolino-quanidine; synthesized using Method C (9% yield)).

Name: N-[4-methyl-6-(4-methylphenoxy)-2-quinazolinyl]guanidine.

Data: ESMS 308 (MH⁺); ¹H NMR (CD₃OD) δ7.89 (d, 1H, J=9.0 Hz), 7.86 (s, 1H), 7.62 (dd, 1H, J=9.0, 2.7 Hz), 7.47 (d, 1H, J=2.4 Hz), 7.23 (d, 2H, J=8.1 Hz), 6.97 (d, 2H, J=8.4 Hz), 2.75 (s, 3H), 2.34 (s, 3H).

### Example 39

N-(6-tert-butyl-4-methyl-2-quinazolinyl)guanidine is made in the same manner as N-(6,7-dibutoxy-4-methyl-2-quinazolinyl)guanidine (see Example 1) except that 6-tert-butylaniline is used in place of 3,4-dibutoxyaniline.

Name: 6-(tert-butyl)-2,2,4-trimethyl-1,2-dihydroquinoline. (synthesized using method B (72% yield).

Data: ESMS 230 (MH⁺); ¹H NMR (CDCl₃) δ6.99 (d, J=7.8 Hz, 1H), 6.66 (dd, J=7.8, 1.5 Hz, 1H), 6.46 (d, J=1.5 Hz, 1H), 5.25 (s, 1H), 3.68 (bs, 1H), 1.97(d, J=1.2 Hz, 3H), 1.28 (d, J=6.0 Hz, 6H), 1.27 (s, 6H).

Compound 1004 (class: Quinazolino-guanidine; synthesized using Method C (45% yield).

Name: N-(6-tert-butyl-4-methyl-2-quinazolinyl)guanidine.

Data: ESMS 258 (MH⁺); ¹H NMR (CD₃OD) δ8.00-8.36 (m, 2H), 7.82 (d, J=8.7 Hz, 1H), 2.90 (s, 3H), 1.42 (s, 9H); Anal. (C₁₄H₁₉N₅. 1.1 CHCl₃. 2.4 NH₃) calcd, C, 42.22; H, 6.40; N, 24.13; Found, C, 42.13; H, 6.36; N, 24.23.

### Example 40

N-(7-ethoxy-4-methyl-2-quinazolinyl)guanidine is made in the same manner as N-(6,7-dibutoxy-4-methyl-2-quinazolinyl)guanidine (see Example 1) except that 3-ethoxyaniline is used in place of 3,4-dibutoxyaniline.

Name: 7-ethoxy-2,2,4-trimethyl-1,2-dihydroquinoline. (synthesized using Method B (37% yield).

Data: ¹H NMR (CDCl₃) δ6.97 (d, J=8.4 Hz, 1H), 6.20 (dd, J=8.4, 2.4 Hz, 1H0, 6.02 (d, J=2.4 Hz, 1H), 5.19 (d, J=1.3 Hz, 1H), 3.98 (q, J=7.0 Hz, 2H), 3.53 (bs, 1H), 1.97 (d, J=1.4 Hz, 3H), 1.39 (t, J=7.0 Hz, 3H), 1.27 (s, 6H).

Compound 1024 (class: Quinazolino-guanidine; synthesized using Method C (42% yield)).

Name: N-(7-ethoxy-4-methyl-2-quinazolinyl)guanidine.

Data: ESMS 244 (MH⁺); ¹H NMR (CD₃OD) δ8.06 (d, J=9.1 Hz, 1H), 7.44 (d, J=2.4 Hz, 1H), 7.31 (dd, J=9.1, 2.5 Hz, 1H), 4.21 (q, J=7.0 Hz, 2H), 2.83 (s, 3H), 1.46 (t, J=7.0 Hz, 3H); Anal. (C₁₂H₁₅N₅O. 1.28 CF₃CO₂H) calcd, C, 44.70; H, 4.19; N, 17.90; Found, C, 44.80; H, 4.09; N, 17.80.

### Example 41

N-[7-(tert-butyl)-4-methyl-2-quinazolinyl]guanidine is made in the same manner as N-(6,7-dibutoxy-4-methyl-2-quinazolinyl)guanidine (see Example 1) except that 3-tert-butylaniline is used in place of 3,4-dibutoxyaniline.

Name: 7-(tert-butyl)-2,2,4-trimethyl-1,2-dihydroquinoline. (synthesized using Method B (82% yield).

Data: ¹H NMR (CDCl₃) δ6.99 (d, J=7.8 Hz, 1H), 6.66 (dd, J=7.8, 1.5 Hz, 1H), 6.46 (d, J=1.5 Hz, 1H), 5.25 (s, 1H) 3.68 (bs, 1H), 1.97(d, J=1.2 Hz, 3H), 1.28 (d, J=6.0 Hz, 6H), 1.27 (s, 6H).

Compound 1022 (class: Quinzolino-guanidine; synthesized using Method C (44% yield)).

Name: N-[7-(tert-butyl)-4-methyl-2-quinazolinyl]guanidine.

Data: ESMS 258 (MH⁺); ¹H NMR (CD₃OD) δ8.09 (d, J=8.7 Hz, 1H), 7.84 (d, J=1.8 Hz, 1H), 7.72 (dd, J=8.7, 1.8 Hz, 1H), 2.86 (s, 3H), 1.41 (s, 9H); mp 195-198° C. (dec.); Anal. (C₁₄H₁₉N₅. 0.9 CH₂Cl₂. 1.2 H₂O. 0.9 NH₃) calcd, C, 48.27; H, 7.04; N, 22.29; Found, C, 47.99; H, 7.04; N, 22.26.

### Example 42

N-(6-hydroxy-4,7-dimethyl-2-quinazolinyl)guanidine is made in the same manner as N-(6,7-dibutoxy-4-methyl-2-quinazolinyl)guanidine (see Example 1) except that 6-nitro-3,4-dihydro-1(2H)-naphthalenone is used in place of 1,2-dibutoxy-4-nitrobenzene.

Name: 6-amino-1,2,3,4-tetrahydro-1-naphthalenol. (synthesized from 6-nitro-3,4-dihydro-1(2H)-naphthalenone using Method A (67% yield).

Data: ESMS 164 (MH⁺); ¹H NMR (CDCl₃) δ6.90 (d, 1H, J=8.1 Hz), 6.79 (d, 1H, J=2.4 Hz), 6.58 (dd, 1H, J=8.1, 2.4 Hz), 4.68 (t, 1H, J=5.4 Hz), 2.68-2.60 (m, 2H), 2.00-1.71 (m, 4H).

Compound 1017 (class: Quinazolino-guanidine; synthesized using methods B & C (28% yield over 2 steps)).

Name: N-(6-hydroxy-4,7-dimethyl-2-quinazolinyl)guanidine.

Data (CF₃CO₂H salt): ESMS 232 (MH⁺); ¹H NMR (CD₃OD) δ7.63 (s, 1H), 7.28 (s, 1H), 2.80 (s, 3H), 2.4 (s, 3H); mp 246-248° C. (dec.); Anal. (CH₁₁H₁₃N₅O. 1.25 CF₃CO₂H. 1 H₂O) calcd, C, 41.39; H, 4.18; N, 17.87; Found, C, 41.52; H, 4.14; N, 17.95.

### Example 43

N-(6-methoxy-4,7-dimethyl-2-quinazolinyl)guanidine is made in the same manner as N-(6,7-dibutoxy-4-methyl-2-quinazolinyl)guanidine (see Example 1) except that 4-methoxyaniline is used in place of 3,4-dibutoxyaniline.

Name: 6-methoxy-2,2,4,7-tetramethyl-1,2-dihydroquinoline. (Synthesized using Method B (82% yield)).

Data: ESMS 218 (MH⁺).

Compound 1016 (class: Quinazolino-guanidine; synthesized using Method C (41% yield)).

Name: N-(6-methoxy-4,7-dimethyl-2-quinazolinyl)guanidine.

Data: ESMS 244 (MH⁺); ¹H NMR (CD₃OD) δ7.63 (s, 1H), 7.30 (s, 1H), 3.98 (s, 3H), 2.86 (s, 3H), 2.39 (s, 3H).

### Example 44

N-(4-methyl-8,9-dihydrobenzo[g]quinazolin-2-yl)guanidine is made in the same manner as N-(6,7-dibutoxy-4-methyl-2-quinazolinyl)guanidine (see Example 1) except that 7-nitro-1-tetralone is used in place of 1,2-dibutoxy-4-nitrobenzene.

Compound 1037 (class: Quinazolino-guanidine; synthesized using Method C (11% yield)).

Name: N-(4-methyl-8,9-dihydrobenzo[g]quinazolin-2-yl)guanidine.

Data: ESMS 254 (MH⁺); ¹H NMR (CD₃OD) δ7.89 (s, 2H), 7.77 (s, 1H), 7.36 (s, 1H), 6.66 (d, 1H, J=9.6 Hz), 6.36 (dt, 1H, J=9.3, 4.5 Hz), 2.97 (br t, 2H), J=7.5 Hz), 2.80 (br s, 3H), 2.45-2.37 (m, 2H).

### Example 45

N-(4-methyl-7,8-dihydro-6H-cyclopenta[g]quinazolin-2-yl)guanidine is made in the same manner as N-(6,7-dibutoxy-4-methyl-2-quinazolinyl)guanidine (see Example 1) except that 5-aminoindane is used in place of 3,4-dibutoxyaniline.

Name: 2,2,4-trimethyl -2,6,7,8-tetrahydro-1H-cyclopenta[g]quinoline (synthesized using Method B (93% yield).

Data: ESMS 214 (MH⁺); ¹H NMR (CDCl₃) δ6.96 (s, 1H), 6.38 (s, 1H), 5.28 (d, 1H, J=0.6 Hz), 2.80 (t, 4H, J=7.2 Hz), 2.16 (br t, 1H, J=7.5 Hz), 2.03 (br t, 1H), 1.99 (br d, 3H, J=0.9 Hz), 1.27 (s, 6H).

Compound 1038 (class: Quinazolino-quanidine; synthesized using Method C (18% yield)).

Name: N-(4-methyl-7,8-dihydro-6H-cyclopenta[g]quinazolin-2-yl)guanidine.

Data: ESMS 242 (MH⁺); ¹H NMR (CD₃OD) δ7.96 (s, 1H), 7.66 (s, 1H), 3.09 (dd, 4H, J=6.9, 6.0 Hz), 2.86 (s, 3H), 2.20 (p, 2H, J=7.5 Hz); mp 295-298° C. (dec.)

### Example 46

N-4-methyl-6-[(5-phenoxypentyl)oxy]-2-quinazolinylguanidine is made in the same manner as N-(6,7-dibutoxy-4-methyl-2-quinazolinyl)guanidine (see Example 1) except that 4-[(5-phenoxypentyl)oxy]aniline is used in place of 3,4-dibutoxyaniline.

Name: 2,2,4-trimethyl-6-[(5-phenoxypentyl)oxy]-1,2-dihydroquinoline (synthesized using Method B).

Data: 352 (ESMS, MH⁺).

Compound 1005 (class: Quinazolino-quanidine; synthesized using Method C (12% yield)).

Name: N-4-methyl-6-[(5-phenoxypentyl)oxy]-2-quinazolinylguanidine.

Data: ESMS 379 (MH⁺); ¹H NMR (CD₃OD) δ7.79 (d, J=9.2 Hz, 1H,), 7.54 (dd, J=9.2, 2.6 Hz, 1H), 7.38 (d, J=2.5 Hz, 1H), 7.21 (t, J=8.0 Hz, 2H), 6.82-6.90 (m, 3H), 4.15 (t, J=6.2 Hz, 2H), 3.98 (t, J=6.2 Hz, 2H), 2.86 (3H, s), 1.62-2.00 (m, 6H).

### Example 47

N-(6-butyl-4-methyl-2-quinazolinyl)guanidine is made in the same manner as N-(6,7-dibutoxy-4-methyl-2-quinazolinyl)guanidine (see Example 1) except that 4-butylaniline is used in place of 3,4-dibutoxyaniline.

Name: 6-butyl-2,2,4-trimethyl-1,2-dihydroquinoline. (synthesized using Method B (14% yield)).

Data: ESMS 230 (MH⁺); ¹H NMR (CDCl₃) δ6.93 (s, 1H), 6.86 (d, 1H, J=8.1 Hz), 6.42 (d, 1H, J=7.8 Hz), 5.35 (br s, 1H), 2.54 (t, 2H, J=7.5 Hz), 2.04 (s, 3H), 1.60 (p, 2H, J=7.5 Hz), 1.40 (septet, 2H, J=7.2 Hz), 1.304 (s, 3H), 1.301 (s, 3H), 0.97 (t, 3H, J=7.2 Hz).

Compound 2004 (class: Quinazolino-guanidine; synthesized using Method C (44% yield)).

Name: N-(6-butyl-4-methyl-2-quinazolinyl)guanidine.

Data: ESMS 258 (MH⁺); ¹H NMR (CD₃OD) δ7.92 (s, 1H, 2^{nd} order coupling), 7.77 (s, 2H, 2^{nd} order coupling), 2.88 (s, 3H), 2.80 (t, 2H, J=7.5 Hz), 1.67 (p, 2H, J=7.8 Hz), 1.39 (septet, 2H, J=7.5 Hz), 0.95 (t, 3H, J=7.2 Hz).

### Example 48

N-(6-benzyl-4-methyl-2-quinazolinyl)guanidine is made in the same manner as N-(6,7-dibutoxy-4-methyl-2-quinazolinyl)guanidine (see Example 1) except that 4-benzylaniline is used in place of 3,4-dibutoxyaniline.

Name: 6-benzyl-2,2,4-trimethyl-1,2-dihydroquinoline. (synthesized using Method B (41% yield)).

Data: ESMS 263 (MH⁺); ¹H NMR (CDCl₃) δ7.14 (t, 2H, J=7.5 Hz), 7.35-7.33 (m, 3H), 7.07 (s, 1H), 6.95 (d, 1H, J=7.8 Hz), 6.51 (dd, 1H, J=8.1, 0.9 Hz), 5.45 (br s, 1H), 4.02 (s, 2H), 2.11 (s, 3H), 1.399 (s, 3H), 1.395 (s, 3H).

Compound 2003 (class: Quinazolino-guanidine; synthesized using Method C (19% yield)).

Name: N-(6-benzyl-4-methyl-2-quinazolinyl)guanidine.

Data: ESMS 298 (MH⁺); ¹H NMR (DMSO-d₆) δ7.62 (br s, 1H), 7.44 (d, 1H, J=8.4 Hz), 7.33 (d, 1H, J=8.1 Hz), 7.22-7.06 (m, 5H), 3.93 (s, 2H), 2.56 (s, 3H).

### Example 49

N-(6-hexyl-4-methyl-2-quinazolinyl)guanidine is made in the same manner as N-(6,7-dibutoxy-4-methyl-2-quinazolinyl)guanidine (see Example 1) except that 4-hexylaniline is used in place of 3,4-dibutoxyaniline.

Name: 6-hexyl-2,2,4-trimethyl-1,2-dihydroquinoline. (synthesized using Method B (32% yield)).

Data: ESMS 258 (MH⁺); ¹H NMR (CDCl₃) δ7.12 (s, 1H), 7.08 (d, 7.8 Hz), 6.55 (dd, 1H, J=7.8, 1.2 Hz), 5.50 (d, 1H, J=1.2 Hz), 2.73 (t, 2H, J=7.2 Hz), 2.21 (d, 3H, J 1.2 Hz), 1.82 (br t, 2H, J=6.0 Hz), 1.55 (br s, 6H), 1.45 (s, 3H), 1.44 (s, 3H), 1.14 (br s, 3H).

Compound 2005 (class: Quinazolino-guanidine; synthesized using Method C (5% yield)).

Name: N-(6-hexyl-4-methyl-2-quinazolinyl)guanidine.

Data: ESMS 286 (MH⁺); ¹H NMR (CD₃OD) δ7.88 (s, 1H), 7.86 (s, 1H, 2^{nd} order coupling), 7.73 (br s, 2H, 2^{nd} order coupling), 2.84 (s, 3H), 2.77 (t, 2H, J=7.8 Hz), 1.6 (br s, 2H), 1.40-1.25 (m, 6H), 0.87 (br t, 3H, J=6.9 Hz).

### Example 50

N-[7-(benzyloxy)-4-methyl-2-quinazolinyl]guanidine is made in the same manner as N-(6,7-dibutoxy-4-methyl-2-quinazolinyl)guanidine (see Example 1) except that 3-(benzyloxy)aniline is used in place of 3,4-dibutoxyaniline.

Name: 7-(benzyloxy)-2,2,4-trimethyl-1,2-dihydroquinoline. (synthesized using Method B (72% yield)).

Data: ¹H NMR (CDCl₃) δ7.34-7.52 (m, 5H), 7.04 (d, J=8.4 Hz, 1H), 6.34 (dd, J=8.4, 2.4 Hz, 1H), 6.16 (d, J=2.4 Hz, 1H), 5.26 (d, J=0.9 Hz, 1H), 5.06 (s, 2H), 3.62 (bs, 1H) 2.02 (d, J=0.9 Hz, 3H), 1.32 (s, 6H).

Compound 1006 (class: Quinazolino-guanidine; synthesized using method C (43% yield)).

Name: N-[7-(benzyloxy)-4-methyl-2-quinazolinyl]guanidine.

Data: ESMS 308 (MS⁺); ¹H NMR (CD₃OD) δ8.01 (d, J=9.0 Hz, 1H), 7.17-7.48 (m, 6H), 7.20 (dd, J=9.0, 2.4 Hz, 1H), 5.20 (s, 2H), 2.78 (s, 3H); mp 215-217° C. (dec.); Anal. (C₁₇H₁₇N₅O.CF₃CO₂H. 0.2 CH₂Cl₂) calcd, C, 52.61; H, 4.23; N, 15.98; Found, C, 52.63; H, 4.26; N, 16.02.

### Example 51

N-(6-heptyl-4-methyl-2-quinazolinyl)guanidine is made in the same manner as N-(6,7-dibutoxy-4-methyl-2-quinazolinyl)guanidine (see Example 1) except that 4-heptylaniline is used in place of 3,4-dibutoxyaniline.

Name: 6-heptyl-2,2,4-trimethyl-1,2-dihydroquinoline. (synthesized using Method B (50% yield)).

Data: ESMS 272 (MH⁺); ¹H NMR (CDCl₃) δ6.89 (dd,1H, J=1.5 Hz), 6.82 (dd, 1H, J=8.1, 2.1 Hz), 5.32 (br s, 1H), 2.49 (br t, 2H, J=7.5 Hz), 2.01 (d, 3H, J=1.2 Hz), 1.60-1.53 (m, 2H), 1.32-1.30 (m, 8H), 1.27 (s, 6H), 0.90 (t, 3H, J=6.9 Hz).

Compound 2006 (class: Quinazolino-guanidine; synthesized using Method C (18% yield)).

Name: N-(6-heptyl-4-methyl-2-quinazolinyl)guanidine.

Data: ESMS 300 (MH⁺); ¹H NMR (DMSO-d₆) δ7.87 (s, 1H), 7.67 (br s, 2H, 2^{nd} order coupling), 2.79 (s, 3H), 2.72 (t, 2H), 1.63 (br s, 2H), 1.30 (br s, 4H), 1.24 (br s, 4H), 0.84 (br t, 3H, J=6.3 Hz).

### Example 52

N-(4-methyl-6-pentyl-2-quinolinyl)guanidine is made in the same manner as N-(6-ethyl-4-methyl-2-quinolinyl)guanidine (see Example 3) except that 4-pentylaniline is used in place of 4-ethylaniline.

Name: 3-oxo-N-(4-pentylphenyl)butanamide. (synthesized from 4-pentylaniline using Method G (28-36% yield).

Data: ESMS 246 (MH⁺); ¹H NMR (CDCl₃) δ9.05 (br s, 1H), 7.43 (d, 2H, J=8.4 Hz), 7.13 (d, 2H, J=8.4 Hz), 3.58 (s, 2H), 2.56 (t, 2H, J=7.5 Hz), 2.32 (s, 3H), 1.58 (p, 2H, J=7.2 Hz), 1.35-1.26(m, 4H), 0.88 (t, 3H, J=6.9 Hz).

Name: 4-methyl-6-pentyl-2(1H)-quinolinone. (synthesized using Method H (76-96% yield)).

Data: ESMS 230 (MH⁺); ¹H NMR (CDCl₃) δ11.92 (br s, 1H), 7.45 (s, 1H, 2^{nd} order coupling), 7.33 (br s, 2H, 2^{nd} order coupling), 6.57 (s, 1H), 2.68 (t, 2H, J=7.8 Hz), 2.51 (s, 3H), 1.64 (br s, 2H), 1.36 (br s, 4H), 0.90 (br s, 3H).

Name: 2-chloro-4-methyl-6-pentylquinoline. (synthesized using Method I (33% yield)).

Data: ESMS 250 & 248 (MH⁺); ¹H NMR (CD₃OD) δ7.83 (br s, 1H), 7.81 (d, 1H, J=8.7 Hz), 7.63 (dd, 1H, J=8.7, 2.1 Hz), 7.33 (d, 1H, J=0.9 Hz), 2.81 (t, 2H, J=7.8 Hz), 2.69 (d, 3H, J=0.9 Hz), 1.71 (br p, 2H, J=7.8 Hz), 1.38-1.33 (m, 4H) 0.90 (br t, 3H, J=6.9 Hz).

Compound 5002 (class: Quinolino-guanidine; synthesized using Method J (2% yield)).

Name: N-(4-methyl-6-pentyl-2-quinolinyl)guanidine.

Data: ESMS 271 (MH⁺); ¹H NMR (CD₃OD) δ7.80 (d, 1H, J=8.4 Hz), 7.75 (d, 1H, J=1.2 Hz), 7.56 (dd, 1H, J=8.4, 1.8 Hz), 6.98 (br s, 1H), 2.78 (dd, 2H, J=7.8, 6.6 Hz), 2.66 (d, 3H, J=0.6 Hz), 1.69 (br p, 2H, J=7.8 Hz), 1.37-1.32 (m, 4H), 0.89 (br t, 3H, J=6.6 Hz).

Example 53

N-(4-methyl-6-propyl-2-quinazolinyl)guanidine is made in the same manner as N-(6,7-dibutoxy-4-methyl-2quinazolinyl)guanidine (see Example 1) except that 4-propylaniline is used in place of 3,4-dibutoxyaniline.

Name: 2,2,4-trimethyl-6-propyl-1,2-dihydroquinoline. (synthesized using Method B (89% yield)).

Data: ESMS 216 (MH⁺); ¹H NMR (CDCl₃) δ6.91 (d, 1H, J=1.8 Hz), 6.84 (dd,1H, J=7.8, 1.8 Hz), 6.41 (d, 1H, J=7.8 Hz), 5.34 (d, 1H, J=1.2 Hz), 2.50 (t, 2H, J=7.5 Hz), 2.02 (d, 3H, J=1.2 Hz), 1.62 (septet, 2H, J=7.8 Hz), 1.29 (s, 6H) 0.96 (t, 3H, J=7.5 Hz).

Compound 1008 (synthesized using Method C (24% yield)).

Name: N-(4-methyl-6-propyl-2-quinazolinyl)guanidine.

Data: ESMS 244 (MH⁺); ¹H NMR (CDCl₃) δ7.64 (s,1H, 2^{nd} order coupling), 7.58 (s, 2H, 2^{nd} order coupling), 2.80 (s, 3H), 2.68 (t, 2H, J=7.2 Hz), 1.65 (septet, 2H, J=7.5 Hz), 0.93 (t, 3H, J=8.4 Hz).

### Example 54

N-(4-methyl-6-phenyl-2-quinazolinyl)guanidine is made in the same manner as N-(6,7-dibutoxy-4-methyl-2-quinazolinyl)guanidine (see Example 1) except that 4-phenylaniline is used in place of 3,4-dibutoxyaniline.

Name: 2,2,4-trimethyl-6-phenyl-1,2-dihydroquinoline. (synthesized using Method B (61% yield)).

Data: ESMS 250 (MH⁺); ¹H NMR (CDCl₃) δ7.77-7.72 (m, 2H), 7.60-7.50 (m, 3H), 7.47-7.40 (m, 2H), 6.65-6.50 (m, 1H), 5.51 (br s, 1H), 2.23 (br s, 3H), 1.44 (br s, 6H).

Compound 1010 (class: Quinazolino-guanidine; synthesized using Method C (3% yield)).

Name: N-(4-methyl-6-phenyl-2-quinazolinyl)guanidine.

Data: ESMS 278 (MH⁺); ¹H NMR (CD₃OD) δ8.31 (d, 1H, J=1.8 Hz), 8.19 (dd, 1H, 8.7, 1.8 Hz), 7.94 (d, 1H, J=8.7 Hz), 7.75 (d, 2H, J=7.2 Hz), 7.50 (t, 2H, J=6.9 Hz), 7.40 (t, 1H, J=7.2 Hz), 2.97 (s, 3H).

### Example 55

N-(4-methyl-6-octyl-2-quinazolinyl)guanidine is made in the same manner as N-(6,7-dibutoxy-4-methyl-2-quinazolinyl)guanidine (see Example 1) except that 4-octylaniline is used in place of 3,4-dibutoxyaniline.

Name: 2,2,4-trimethyl-6-octyl-1,2-dihydroquinoline. (synthesized using Method B (72% yield)).

Data: ESMS 286 (MH⁺); ¹H NMR (CDCl₃) δ6.90-6.75(m, 2H), 6.41-6.33 (m, 1H), 5.29 (br s, 1H), 2.50-2.42 (m, 2H), 2.01-1.96 (m, 3H), 1.55 (br s, 2H), 1.29-1.21 (m, 16H), 0.91-0.54 (m, 3H).

Compound 1009 (class: Quinazolino-guanidine; synthesized using Method C (12% yield)).

Name: N-(4-methyl-6-octyl-2-quinazolinyl)guanidine.

Data: ESMS 314 (MH⁺); ¹H NMR (DMSO-d₆) δ7.79 (s, 1H, 2^{nd} order coupling), 7.62-7.50 (m, 2H, 2^{nd} order coupling), 2.732 (br s, 5H), 1.60 (br s, 2H), 1.21 (br s, 10H), 0.82 (br t, 3H).

### Example 56

N-(6-hexyl-4-methyl-2-quinolinyl)guanidine is made in the same manner as N-(6-ethyl-4-methyl-2-quinolinyl)guanidine (see Example 3) except that 4-hexylaniline is used in place of 4-ethylaniline.

Name: N-(4-hexylphenyl)-3-oxobutanamide. (synthesized from 4-hexylaniline using Method G (54% yield)).

Name: 6-hexyl-4-methyl-2(1H)-quinolinone. (synthesized using Method H (100% yield)).

Data: ESMS 244 (MH⁺).

Name: 2-chloro-6-hexyl-4-methylquinoline. (synthesized using Method I (60% yield)).

Data: ESMS 264 & 262 (MH⁺); ¹H NMR (CDCl₃) δ7.78 (br d, 1H, J=2.4 Hz), 7.75 (s, 1H), 7.59 (dd, 1H, J=8.7, 1.5 Hz), 7.27 (br s, 1H), 2.77 (t, 2H, J=7.5 Hz), 2.64 (s, 3H), 1.67 (br p, 2H, J=7.2 Hz), 1.31 (br s, 6H), 0.86 (br t, 3H, J=6.9 Hz).

Compound 5003 (class: Quinolino-guanidine; synthesized using Method J (10% yield)).

Name: N-(6-hexyl-4-methyl-2-quinolinyl)guanidine.

Data: ESMS 285 (MH⁺); ¹H NMR (CD₃OD) δ7.72 (d, 1H, J=8.7 Hz), 7.67 (d, 1H, J=0.9 Hz), 7.51 (dd, 1H, J=8.4, 1.8 Hz), 6.92 (br s, 1H), 2.75 (t, 2H, J=7.5 Hz), 2.60 (s, 3H), 1.67 (br p, 2H, J=7.8 Hz), 1.32 (br s, 6H), 0.88 (br t, 3H, J=6.9 Hz).

### Example 57

N-(6-[1-(4-hydroxyl-pentyl)]-4-methyl-2-quinazolino)guanidine is made in the same manner as N-(6-ethyl-4-methyl-2-quinazolino)guanidine (see Example 1) except that 5-(4-aminophenyl)-2-pentanol is used in place of 4-ethylaniline.

Compound 1034

Name: N-(6-[1-(4-hydroxyl-pentyl)]-4-methyl-2-quinazolino)guanidine.

Data: ESMS 288 (MH⁺); ¹H NMR (CD₃OD) δ7.96 (s, 1H), 7.80 (s, 2H), 3.74 (p, J=6.3 Hz, 1H), 2.90 (s, 3H), 2.85-2.81 (m, 2H), 1.85-1.65 (m, 2H), 1.55-1.45 (m, 2H), 1.14 (d, J=6.3 Hz, 3H).

### Example 58

N-(6-butyl-4-methyl-2-quinolinyl)guanidine is made in the same manner as N-(6-ethyl-4-methyl-2-quinolinyl)guanidine (see Example 3) except that 4-butylaniline is used in place of 4-ethylaniline.

Compound 5001

Name: N-(6-butyl-4-methyl-2-quinolinyl)guanidine.

Data: ESMS 257 (MH⁺); ¹H NMR (CD₃OD) δ7.82 (d, J=8.4 Hz, 1H), 7.78 (d, J=1.5 Hz, 1H), 7.58 (dd, J=8.4, 1.5 Hz, 1H), 6.93 (s, 1H), 2.81 (t, J=7.2 Hz, 2H), 2.68 (s, 3H), 1.69 (p, J=7.2 Hz, 2H), 1.39 (sextet, J=7.2 Hz, 2H), 0.95 (t, J=7.2 Hz, 3H).

### Example 59

N-(4-methyl-7-phenyl-2-quinazolinyl)guanidine is made in the same manner as N-(6,7-dibutoxy-4-methyl-2-quinazolinyl)guanidine (see Example 1) except that 3-phenylaniline is used in place of 3,4-dibutoxyaniline.

Compound 1023

Name: N-(4-methyl-7-phenyl-2-quinazolinyl)guanidine.

Data: ESMS 278 (MH⁺); ¹H NMR (CD₃OD) δ8.17 (br s, 1H), 8.05 (br s, 1H), 7.84 (br s, 1H), 7.70 (br s, 2H), 7.43 (br s, 2H), 7.35 (br s, 1H), 2.87 (s, 3H).

### Example 60

N-[4-methyl-7-(isopropoxy)-2-quinazolinyl]guanidine is made in the same manner as N-(6,7-dibutoxy-4-methyl-2-quinazolinyl)guanidine (see Example 1) except that 3-isopropoxyaniline is used in place of 3,4-dibutoxyaniline.

Compound 1025

Name: N-[4-methyl-7-(isopropoxy)-2-quinazolinyl]guanidine.

Data: ESMS 260 (MH⁺); ¹H NMR (CD₃OD) ? 8.03 (d, J=9.3 Hz, 1H), 7.23 (d, J=2.4 Hz, 1H), 7.13 (dd, J=9.3, 2.4 Hz, 1H), 3.29 (septet, J=6.0 Hz, 1H), 2.81 (s, 3H), 1.39 (d, J=6.0 Hz, 6H).

Table 1. Summary of the compounds prepared.

TABLE 1

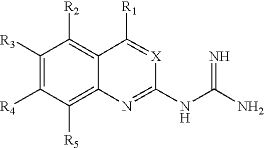

| Com- | | | | | | |
|---|---|---|---|---|---|---|
| pound | X | R₁ | R₂ | R₃ | R₄ | R₅ |
| 1001 | N | meth- | H | H | H | H |
| | | yl | | | | |
| 1002 | N | meth- | H | methyl | H | H |
| | | yl | | | | |
| 1003 | N | meth- | H | ethyl | H | H |
| | | yl | | | | |
| 1004 | N | meth- | H | tert-butyl | H | H |
| | | yl | | | | |
| 1005 | N | meth- | H | 5-phenoxy- | H | H |
| | | yl | | pentyloxy | | |
| 1006 | N | meth- | H | H | benzyloxy | H |
| | | yl | | | | |
| 1007 | N | meth- | fused benzene | | H | H |
| | | yl | | | | |
| 1008 | N | meth- | H | propyl | H | H |
| | | yl | | | | |
| 1009 | N | meth- | H | octyl | H | H |
| | | yl | | | | |
| 1010 | N | meth- | H | phenyl | H | H |
| | | yl | | | | |
| 1011 | N | meth- | H | OMe | H | H |
| | | yl | | | | |
| 1012 | N | meth- | H | OBu | H | H |
| | | yl | | | | |
| 1013 | N | meth- | H | Cl | H | H |
| | | yl | | | | |
| 1014 | N | meth- | H | H | Br | H |
| | | yl | | | | |
| 1015 | N | meth- | H | methyl | methyl | H |
| | | yl | | | | |
| 1016 | N | meth- | H | OMe | methyl | H |
| | | yl | | | | |
| 1017 | N | meth- | H | OH | methyl | H |
| | | yl | | | | |
| 1018 | N | meth- | H | OBu | OBu | H |
| | | yl | | | | |
| 1019 | N | meth- | H | F | F | H |
| | | yl | | | | |
| 1020 | N | meth- | H | H | ethyl | H |
| | | yl | | | | |
| 1021 | N | meth- | H | H | isopropyl | H |
| | | yl | | | | |
| 1022 | N | meth- | H | H | tert-butyl | H |
| | | yl | | | | |
| 1023 | N | meth- | H | H | phenyl | H |
| | | yl | | | | |
| 1024 | N | meth- | H | H | OEt | H |
| | | yl | | | | |
| 1025 | N | meth- | H | H | isopropoxy | H |
| | | yl | | | | |
| 1026 | N | meth- | H | Br | H | H |
| | | yl | | | | |
| 1027 | N | ethyl | H | methyl | H | H |
| 1028 | N | meth- | H | benzyloxy | H | H |
| | | yl | | | | |
| 1029 | N | meth- | H | cyclohexyl | H | H |
| | | yl | | | | |
| 1030 | N | meth- | H | OCF₃ | H | H |
| | | yl | | | | |
| 1031 | N | meth- | H | pentyloxy | H | H |
| | | yl | | | | |
| 1032 | N | meth- | H | OPh | H | H |
| | | yl | | | | |
| 1033 | N | meth- | H | 4-methylphenyloxy | H | H |
| | | yl | | | | |
| 1034 | N | meth- | H | 4-hydroxypentyl | H | H |
| | | yl | | | | |
| 1035 | N | meth- | H | H | 1-hydroxy- | H |
| | | yl | | | ethyl | |
| 1036 | N | meth- | H | H | OCF₃ | H |
| | | yl | | | | |
| 1037 | N | meth- | H | fused 5,6- cyclohexenyl | | H |
| | | yl | | | | |
| 1038 | N | meth- | H | fused cyclopentyl | | H |
| | | yl | | | | |
| 1039 | N | meth- | H | fused 2,3-furyl | | H |
| | | yl | | | | |
| 2001 | N | meth- | H | pentyl | H | H |
| | | yl | | | | |
| 2002 | N | meth- | H | sec-butyl | H | H |
| | | yl | | | | |
| 2003 | N | meth- | H | benzyl | H | H |
| | | yl | | | | |
| 2004 | N | meth- | H | butyl | H | H |
| | | yl | | | | |
| 2005 | N | meth- | H | hexyl | H | H |
| | | yl | | | | |
| 2006 | N | meth- | H | heptyl | H | H |
| | | yl | | | | |
| 3001 | N | meth- | H | H | methyl | H |
| | | yl | | | | |
| 4001 | C | meth- | H | methyl | H | H |
| | | yl | | | | |
| 4002 | C | meth- | H | ethyl | H | H |
| | | yl | | | | |
| 4003 | C | meth- | H | Ph | H | H |
| | | yl | | | | |
| 4004 | C | meth- | H | OMe | H | H |
| | | yl | | | | |
| 4005 | C | meth- | H | Cl | H | H |
| | | yl | | | | |
| 4006 | C | meth- | H | H | methyl | H |
| | | yl | | | | |
| 4007 | C | meth- | H | H | F | H |
| | | yl | | | | |
| 4008 | C | meth- | meth- | H | methyl | H |
| | | yl | yl | | | |
| 4009 | C | meth- | fused benzene | | H | H |
| | | yl | | | | |
| 5001 | C | meth- | H | butyl | H | H |
| | | yl | | | | |
| 5002 | C | meth- | H | pentyl | H | H |
| | | yl | | | | |
| 5003 | C | meth- | H | hexyl | H | H |
| | | yl | | | | |
| 6001 | C | meth- | H | H | H | H |
| | | yl | | | | |
| 6002 | C | meth- | H | H | H | methyl |
| | | yl | | | | |
| 6003 | C | ethyl | H | H | methyl | H |

II. Testing of Chemical Compounds

Test 1

The binding properties of the compounds of the present invention were evaluated at cloned NPFF receptors using protocols described in PCT International Publication No. WO 00/18438, the disclosure of which is hereby incorporated by reference in its entirety into this application.

TABLE 2
Binding affinities at Recombinant Human and Rat NPFF Receptors

| | hNPFF1 | hNPFF2 | rNPFF1 | rNPFF2 |
|---|---|---|---|---|
| Compound | Ki (nM) | Ki (nM) | Ki (nM) | Ki (nM) |
| 3001 | 46 | 1,717 | 50 | 1,222 |
| 1001 | 240 | 2,043 | 202 | >10,000 |
| 1007 | 53 | 260 | 146 | 699 |
| 6001 | 23 | 374 | 11 | 433 |
| 4006 | 13 | 91 | 7 | 185 |
| 6003 | 28 | 113 | 21 | 203 |
| 6002 | 157 | 952 | 91 | 883 |
| 4005 | 24 | 123 | 25 | 282 |
| 4009 | 144 | 826 | 153 | 871 |
| 4004 | 113 | 1,214 | 153 | 2,584 |
| 4008 | 82 | 514 | 64 | 882 |
| 4001 | 21 | 150 | 30 | 556 |
| 4003 | 207 | 2,125 | 176 | 1,252 |
| 1020 | NT | NT | 18 | 273 |
| 4007 | NT | NT | 44 | 619 |
| 1002 | NT | NT | 134 | 3,919 |
| 1019 | NT | NT | 57 | 2,874 |
| 1014 | NT | NT | 300 | 3,439 |
| 1026 | NT | NT | 802 | >10,000 |
| 1036 | NT | NT | 132 | 2,458 |
| 1013 | NT | NT | 332 | 2,019 |
| 1011 | NT | NT | 201 | >10,000 |
| 1021 | NT | NT | 56 | 881 |
| 1030 | NT | NT | 176 | 4,864 |
| 2001 | 50 | 376 | 8 | 221 |
| 1015 | NT | NT | 42 | 1,108 |
| 1035 | NT | NT | 842 | 1,183 |
| 1003 | NT | NT | 238 | 1,638 |
| 2002 | NT | NT | 77 | 461 |
| 1039 | NT | NT | 68 | 2,930 |
| 4002 | 50 | 232 | 11 | 308 |
| 1012 | NT | NT | 733 | 4,845 |
| 1028 | NT | NT | 386 | 817 |
| 1032 | NT | NT | 291 | 1,638 |
| 1029 | NT | NT | 912 | 1,201 |
| 1031 | NT | NT | 794 | 3,223 |
| 1033 | NT | NT | 481 | 5,864 |
| 1004 | NT | NT | 710 | 1,488 |
| 1016 | NT | NT | 565 | 2,496 |
| 1024 | NT | NT | 659 | 5,593 |
| 1018 | NT | NT | 303 | 1,299 |
| 1022 | NT | NT | 126 | 602 |
| 1017 | NT | NT | 234 | 5,919 |
| 1037 | NT | NT | 143 | 824 |
| 1008 | NT | NT | 155 | 1,121 |
| 1038 | NT | NT | 95 | 602 |
| 1005 | NT | NT | 316 | 2,138 |
| 2004 | NT | NT | 392 | 262 |
| 2003 | NT | NT | 371 | 195 |
| 2005 | NT | NT | 88 | 268 |
| 1006 | NT | NT | 410 | 1,071 |
| 1010 | NT | NT | 311 | 3,480 |
| 1009 | NT | NT | 312 | 703 |
| 2006 | NT | NT | 788 | 3,674 |
| 5002 | 40 | 460 | 30 | 569 |
| 5003 | 152 | 1,172 | 532 | 4,423 |
| 1034 | NT | NT | 82 | 1,537 |
| 5001 | NT | NT | 24 | 115 |
| 1023 | 228 | 2,919 | 4 | 1,019 |
| 1025 | NT | NT | 253 | 4,534 |
| 1027 | NT | NT | 606 | 3,154 |

NT = Not Tested

Test 2

Activity of the compounds of the present invention was measured at cloned NPFF receptors according to functional assays as previously described by Bonini, J. A., et al. (3). Agonist potency (EC₅₀) is the concentration of a compound required to elicit 50% of maximum response. Intrinsic activity of a compound is measured as the percent of maximum response. Intrinsic activity of a compound is measured as the percent of maximum response elicited by the ligand, neuropeptide FF.

TABLE 3
Agonist Potency (EC₅₀) and Intrinsic Activity (IA) at
Recombinant Human (3-1) and Rat (3-2) Neuropeptide FF Receptors
Table 3-1.

| | hNPFF1 | hNPFF1 | hNPFF2 | hNPFF2 |
|---|---|---|---|---|
| Compound | EC₅₀ (nM) | IA (% NPFF) | EC₅₀ (nM) | IA (% NPFF) |
| 3001 | >10,000 | Inactive | >10,000 | Inactive |
| 6001 | >10,000 | Inactive | >10,000 | Inactive |
| 4006 | >10,000 | Inactive | >10,000 | Inactive |
| 2001 | 3,453 | Inactive | 625 | 84% |
| 4002 | >10,000 | Inactive | 314 | 69% |
| 5002 | >10,000 | Inactive | 1,707 | 75% |
| 5003 | >10,000 | Inactive | 3,160 | 45% |
| 1023 | >10,000 | Inactive | 4,114 | 43% |

Table 3-2.

| | rNPFF1 | rNPFF1 | rNPFF2 | rNPFF2 |
|---|---|---|---|---|
| Compound | EC₅₀ (nM) | IA (% NPFF) | EC₅₀ (nM) | IA (% NPFF) |
| 1001 | >10,000 | Inactive | 3,084 | 16% |
| 1007 | >10,000 | Inactive | 1,296 | 66% |
| 6001 | >10,000 | Inactive | >10,000 | Inactive |
| 4006 | >10,000 | Inactive | 269 | 32% |
| 6003 | >10,000 | Inactive | >10,000 | Inactive |
| 6002 | >10,000 | Inactive | >10,000 | Inactive |
| 4005 | >10,000 | Inactive | 389 | 61% |
| 4009 | >10,000 | Inactive | 3,160 | 70% |
| 4004 | >10,000 | Inactive | 1,528 | 65% |
| 4008 | >10,000 | Inactive | 411 | 65% |
| 4001 | >10,000 | Inactive | 404 | 68% |
| 4003 | >10,000 | Inactive | 3,160 | 26% |
| 1020 | >10,000 | Inactive | 695 | 90% |
| 4007 | >10,000 | Inactive | 2,637 | 17% |
| 1002 | >10,000 | Inactive | 5,621 | 24% |
| 1019 | >10,000 | Inactive | 2,543 | 31% |
| 1014 | >10,000 | Inactive | 2,462 | 47% |
| 1026 | >10,000 | Inactive | >10,000 | 19% |
| 1036 | >10,000 | Inactive | 369 | 78% |
| 1013 | >10,000 | Inactive | 690 | 52% |
| 1011 | >10,000 | Inactive | >10,000 | Inactive |
| 1021 | >10,000 | Inactive | 283 | 76% |
| 1030 | >10,000 | Inactive | 625 | 85% |
| 2001 | 242 | 71% | 97 | 103% |
| 1015 | >10,000 | Inactive | 272 | 56% |
| 1035 | >10,000 | Inactive | 3,160 | 52% |
| 1003 | >10,000 | Inactive | 392 | 83% |
| 2002 | 250 | 51% | 423 | 92% |
| 1039 | >10,000 | Inactive | 272 | 78% |
| 4002 | >10,000 | Inactive | 125 | 84% |
| 1012 | >10,000 | Inactive | 1,616 | 80% |
| 1028 | >10,000 | Inactive | 758 | 79% |
| 1032 | 374 | 31% | 459 | 93% |
| 1029 | >10,000 | 28% | 2,046 | 31% |
| 1031 | >10,000 | Inactive | 2,187 | 66% |
| 1033 | >10,000 | Inactive | 3,160 | 51% |
| 1004 | 1,469 | 36% | 440 | 90% |
| 1016 | >10,000 | Inactive | 3,160 | 74% |
| 1024 | >10,000 | Inactive | >10,000 | Inactive |
| 1018 | >10,000 | Inactive | >10,000 | Inactive |
| 1022 | 3,160 | 19% | 190 | 81% |
| 1017 | >10,000 | Inactive | >10,000 | 23% |
| 1037 | >10,000 | Inactive | 3,160 | 71% |
| 1008 | >10,000 | Inactive | 619 | 85% |
| 1038 | >10,000 | Inactive | 48 | 74% |
| 1005 | >10,000 | Inactive | 3,160 | 21% |
| 2004 | 194 | 40% | 124 | 101% |
| 2003 | 171 | 56% | 49 | 89% |
| 2005 | 137 | 56% | 105 | 81% |
| 1006 | >10,000 | 15% | 1,080 | 22% |
| 1010 | >10,000 | Inactive | >10,000 | 22% |
| 1009 | 1,494 | Inactive | 5,621 | 22% |
| 2006 | 886 | 38% | 1,953 | 47% |
| 5002 | 157 | 41% | 259 | 90% |
| 5003 | 440 | 27% | 9,993 | 57% |
| 1034 | 610 | 63% | 394 | 101% |
| 5001 | 123 | 28% | 69 | 82% |
| 1023 | >10,000 | Inactive | 3,160 | 35% |
| 1025 | >10,000 | Inactive | 3,160 | 27% |
| 1027 | >10,000 | Inactive | >10,000 | 31% |

Test 3

Methods for two NPFF2 selective compounds that were tested in vivo experiment

The effects of compounds on the micturition reflex were assessed in the “distension-induced rhythmic contraction” (DIRC) model (also called “volume-induced rhythmic contraction” model) in rats, as described in previous publications (36, 38, 40). This model is widely considered to be predictive for the actions of drugs to treat human urge incontinence (also referred to as detrusor instability or unstable bladder). Examples of drugs that are active in this model which also are used therapeutically in humans include oxybutynin and baclofen (40); imipramine and nortriptyline (37); and nifedipine and terodiline (38).

DIRC Model

Female Sprague Dawley rats weighing approximately 300 g were anesthetized with subcutaneous urethane (1.2 g/kg) The trachea was cannulated with PE240 tubing to provide a clear airway throughout the experiment. A midline abdominal incision was made and the left and right ureters were isolated. The ureters were ligated distally (to prevent escape of fluids from the bladder) and cannulated proximally with PE10 tubing. The incision was closed using 4-0 silk sutures, leaving the PE10 lines routed to the exterior for the elimination of urine. The bladder was canulated via the transurethral route using PE50 tubing inserted 2.5 cm beyond the urethral opening. This cannula was secured to the tail using tape and connected to a pressure transducer. To prevent leakage from the bladder, the cannula was tied tightly to the exterior urethral opening using 4-0 silk.

To initiate the micturition reflex, the bladder was first emptied by applying pressure to the lower abdomen, and then filled with normal saline in 100 μL increments (maximum=2 ml) until spontaneous bladder contractions occurred (typically 20-40 mmHg) at a rate of one contraction every 2 to 3 minutes. Once a regular rhythm was established, vehicle (saline) or test compounds were administered i.v. to examine their effects on bladder activity. The effect of a compound which inhibited the micturition reflex was expressed as its “disappearance time”, defined as the time between successive bladder contractions in the presence of the test compound minus the time between contractions before compound administration.

Results of Test 3

Compound X (4005) at a dose of 1 mg/kg, i.v. produced complete inhibition of distention induced contractions of the rat bladder, resulting in a disappearance time of 35 minutes. Compound Y (4006) at a dose of 3 mg/kg, i.v. produced complete inhibition of distention induced contractions of the rat bladder, resulting in a disappearance time of 12 minutes.

Discussion of Test 3

These results represent the first demonstration that synthetic ligands which are active as agonists at the NPFF2 receptor inhibit the micturition reflex. In this regard their actions mimic the action of the endogenous peptide ligand NPFF. The ability of these compounds to inhibit the micturition reflex in this model can be taken as an indication that they will be effective in the treatment of urge incontinence in humans (see above).

### DISCUSSION

The compounds discussed above can be classified as agonists and antagonists based on the following parameters: an agonist has an intrinsic activity (IA)>15%, while an antagonist has a Ki≦1.2 μM and an intrinsic activity (IA)≦15% at the rat cloned neuropeptide FF (NPFF) receptors.

Based on this definition the compounds can be classified as follows:

Compounds 1001 to 1039 are quinazolino-guanidines that are antagonists at NPFF1 and agonists at NPFF2;

Compounds 2001 to 2006 are quinazolino-guanidines that are concurrently agonists at NPFF1 and NPFF2;

Compound 3001 is quinazolino-guanidines that is concurrently antagonists at NPFF1 and NPFF2;

Compounds 4001 to 4009 are quinolino-guanidines that are antagonists at NPFF1 and agonists at NPFF2;

Compounds 5001 to 5003 are quinolino-quanidines that are concurrently agonists at NPFF1 and NPFF2; and

Compounds 6001 to 6003 are quinolino-quanidines that are concurrently antagonists at NPFF1 and NPFF2.

Compounds that are agonists at NPFF2 are suitable for treating incontinence, and also pain.

Compounds that are concurrently agonists at both NPFF1 and NPFF2 are particularly suitable for treating incontinence, and also pain.

Compounds that are concurrently antagonists at both NPFF1 and NPFF2 have a pro-opioid (analgesic) effect.

Compounds that are agonists at NPFF1 are suitable for treating obesity or eating disorders.

When comparing the binding affinities of compounds between the human and rat recombinant NPFF receptors, one obtains a positive correlation with slope values close to unity, the line of identity. These data suggest that the binding affinity for a compound at the rat receptor will be predictive of its binding affinity at the human recombinant receptor.

### REFERENCES

1. Yang, H. Y., Fratta, W., Majane, E. A., and Costa, E. Isolation, sequencing, synthesis, and pharmacological characterization of two brain neuropeptides that modulate the action of morphine. Proc.Natl.Acad.Sci.U.S.A. 82(22):7757-7761, 1985.

2. Vilim, E. S., Ziff, E. Cloning of the neuropeptide NPFF and NPAF precursor form bovine, rat, mouse, and human. Soc. Neurosci. 21:760, 1995.

3. Bonini, J. A., Jones, K. A., Adham, N., Forray, C., Artymyshyn, R., Durkin, M. M., Smith, K. E., Tamm, J. A., Boteju, L. W., Lakhlani, P. P., Raddatz, R., Yao, W-J., Ogozaleck, K. L., Boyle, N., Kouranova, E. V., Quan, Y., Vyase, P. J., Wetzel, J. M., Branchek, T. A., Gerald, C., Borowsky, B. Identification and characterization of two G protein-coupled receptors for neuropeptide FF. J. Biol. Chem. 275(50): 39324-31, 2000.

4. DNA Encoding Mammalian Neuropeptide FF (NPFF) Receptors and Uses Thereof, PCT International Publication No. WO 00/18438.

5. Panula, P., Aarnisalo, A. A., and Wasowicz, K. Neuropeptide FF, a mammalian neuropeptide with multiple functions [published erratum appears in Prog. Neurobiol. 1996 Jun; 49(3):285]. Prog. Neurobiol. 48(4-5):461-487, 1996.

6. Roumy, M. and Zajac, J. M. Neuropeptide FF, pain and analgesia. Eur.J.Pharmacol. 345(1):1-11, 1998.

7. Payza, K., Akar, C. A., and Yang, H. Y. Neuropeptide FF receptors: structure-activity relationship and effect of morphine. J.Pharmacol.Exp.Ther. 267(1):88-94, 1993.

8. Raffa, R. B., Kim, A., Rice, K. C., de Costa, B. R., Codd, E. E., and Rothman, R. B. Low affinity of FMRFamide and four FaRPs (FMRFamide-related peptides), including the mammalian-derived FaRPs F-8-Famide (NPFF) and A-18-Famide, for opioid mu, delta, kappa 1, kappa 2a, or kappa 2b receptors. Peptides 15(3):401-404, 1994.

9. Malin, D. H., Lake, J. R., Arcangeli, K. R., Deshotel, K. D., Hausam, D. D., Witherspoon, W. E., Carter, V. A., Yang, H. Y., Pal, B., and Burgess, K. Subcutaneous injection of an analog of neuropeptide FF precipitates morphine abstinence syndrome. Life Sci. 53(17):PL261-6, 1993.

10. Malin, D. H., Lake, J. R., Fowler, D. E., Hammond, M. V., Brown, S. L., Leyva, J. E., Prasco, P. E., and Dougherty, T. M. FMRF-NH2-like mammalian peptide precipitates opiate-withdrawal syndrome in the rat. Peptides 11(2):277-280, 1990.

11. Panula, P., Kivipelto, L., Nieminen, O., Majane, E. A., and Yang, H. Y. Neuroanatomy of morphine-modulating peptides. Med.Biol. 65(2-3):127-135, 1987.

12. Allard, M., Geoffre, S., Legendre, P., Vincent, J. D., and Simonnet, G. Characterization of rat spinal cord receptors to FLFQPQRFamide, a mammalian morphine modulating peptide: a binding study. Brain Res. 500(1-2):169-176, 1989.

13. Allard, M., Zajac, J. M., and Simonnet, G. Autoradiographic distribution of receptors to FLFQPQRFamide, a morphine-modulating peptide, in rat central nervous system. Neuroscience 49(l):101-116, 1992.

14. Gouarderes, C., Tafani, J. A. M., and Zajac, J. M. Affinity of neuropeptide FF analogs to opioid receptors in the rat spinal cord. Peptides 19(4):727-730, 1998.

15. Payza, K. and Yang, H. Y. Modulation of neuropeptide FF receptors by guanine nucleotides and cations in membranes of rat brain and spinal cord. J.Neurochem. 60(5):1894-1899, 1993.

16. Gouarderes, C., Sutak, M., Zajac, J. M., and Jhamandas, K. Antinociceptive effects of intrathecally administered F8Famide and FMRFamide in the rat. Eur.J.Pharmacol. 237(1):73-81, 1993.

17. Yang, H. Y. T., Martin, B. M. Soc. Neurosci. 21, 760, 1995.

18. Jhamandas, K., Sutak, M., Yang, H.-Y. T. Soc. Neurosci. 22, 1313, 1996.

19. Kontinen, V. K., Aarnisalo, A. A., Idaenpaeaen-Heikkilae, J. J., Panula, P., and Kalso, E. Neuropeptide FF in the rat spinal cord during carrageenan inflammation. Peptides 18(2):287-292, 1997.

20. Wei, H., Panula, P., and Pertovaara, A. A differential modulation of allodynia, hyperalgesia and nociception by neuropeptide FF in the periaqueductal gray of neuropathic rats: Interactions with morphine and naloxone. Neuroscience 86(1):311-319, 1998.

21. Oberlin P., Stinus, L., Le Moal, M., and Simonnet, G. Biphasic effect on nociception and antiopiate activity of the neuropeptide FF (FLFQPQRFamide) in the rat. Peptides 14(5):919-924, 1993.

22. Gouarderes, C., Jhamandas, K., Sutak, M., and Zajac, J. M. Role of opioid receptors in the spinal antinociceptive effects of neuropeptide FF analogues. Br.J.Pharmacol. 117(3):493-501, 1996.

23. Gouarderes, C., Kar, S., Zajac, J.-M., Neuroscience, 74, 21-27, 1996.

24. Xu, M.; Kontinen, V. K.; Panula, P.; Kalso, E. Peptides, 10, 1071-1077, 1999.

25. Coudoré, M. A.; Courteix, C.; Eschalier, A.; Zajac, J.-M., Wilcox, G. L.; Fialip, J. Resumes de la 1ere Reunion de la Societe Francaise de Pharmacologie (Marseilles, France), vol. 17, p23, 1997.

26. Huang, E. Y.-K.; Li, J. Y.; Tan, P. P.-C.; Wong, C.-H.; Chen, J.-C. Peptides, 21, 205-210, 2000.

27. Robert, J. J., Orosco, M., Rouch, C., Jacquot, C., and Cohen, Y. Unexpected responses of the obese “cafeteria” rat to the peptide FMRF-amide. Pharmacol.Biochem.Behav. 34(2):341-344, 1989.

28. Murase, T., Arima, H., Kondo, K., and Oiso, Y. Neuropeptide FF reduces food intake in rats. Peptides 17(2):353-354, 1996.

29. Kavaliers, M., Hirst, M., and Mathers, A. Inhibitory influences of FMRFamide on morphine- and deprivation-induced feeding. Neuroendocrinology. 40(6):533-535, 1985.

30. Payza, K. and Yang, H. Y. Modulation of neuropeptide FF receptors by guanine nucleotides and cations in membranes of rat brain and spinal cord. J.Neurochem. 60(5):1894-1899, 1993.

31. Devillers, J. P., Mazarguil, H., Allard, M., Dickenson, A. H., Zajac, J. M., and Simonnet, G. Characterization of a potent agonist for NPFF receptors: binding study on rat spinal cord membranes. Neuropharmacology 33(5):661-669, 1994.

32. Cowan, J. A., (1986) “Cu2+/BH4-Reduction System: Synthetic Utility And Mode of Action”, Tetrahedron Lett 27 pages 1205-1208.

33. a) Brown, J. P., (1964) “Reactions of 2,2-Dialkyl-1,2-dihydroquinolines, Part I. Preparation of 2-Guanidinoquinazolines”, J. Chem. Soc. Pages 3012-3016 b) Hamann, L. G., et al, (1998) “Synthesis and Biological Activity of a Novel Series of Nonsteroidal,Peripherally Selective Androgen Receptor Antagonists Derived from 1,2-Dihydropyridono[5,6-g]quinolines”, J. Med. Chem. 41 pages 623-639.

34. Hynes, J. B. and Campbell, J. P., (1997) “2-Amino-quinazolines”, J. Heterocycl. Chem. 34(2) pages 385-387.

35. Kuhla, D. E., et al, (1986) “Quinoline and Quinazoline Derivatives for Treating Gastrointestinal Motility Dysfunctions”, U.S. Pat. No. 4,563,460.

36. Maggi, C. A, Furio, M., Santicioli, P., Conte, B. and Meli, A. (1987) “Spinal and supraspinal components of GABAergic inhibition of the micturition reflex in rats. J. Pharmacol Exp Ther 240, 998-1005.

37. Pietra, C., Poggesi, E., Angelico, P., Guarneri, L. and Testa, R. Effects of some antidepressants on the volume-induced reflex contractions of the rat urinary bladder: lack of correlation with muscarinic receptors affinity. Pharmacological Research, 22: 421-432, 1990.

38. Guarneri, L., Ibba, M., Angelico, P., Colombo, D., Fredella, B. and Testa, R. Effects of drugs used in the therapy of detrusor hyperactivity on the volume-induced contractions of the rat urinary bladder. Pharmacological Research, 27: 173-187, 1993.

39. Morgan, D. G., Small, C. J., Abusnana, S., Turton, M., Gunn, I., Heath, M., Rossi, M., Goldstone, A. P., O'Shea, D., Meeran, K., Ghatei, M., Smith, D. M., and Bloom, S. The NPY Y1 receptor antagonist BIBP 3226 blocks NPY induced feeding via a non-specific mechanism. Regul. Pept. 75-76: 377-382, 1998.

40. Morikawa, K., Hashimoto, S., Yamauchi, T., Kato, H., Ito, Y., and Gomi, Y. (1992) “Inhibitory effect of inaperisone hydrochloride (inaperisone), a new centrally acting muscle relaxant, on the micturition reflex” Eur J pharmacol 213, 409-415.

What is claimed is:

1. A method of treating urge incontinence in a subject in need of such treatment comprising administering to the subject an effective amount of a compound having the structure: 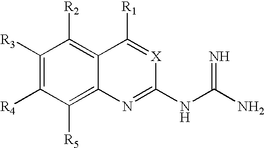
wherein X=CH, C(CH₃) or N;
wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;
and wherein each alkyl, alkenyl, alkynyl and alkoxy group is optionally substituted with a substituent independently selected from Rₐ where Rₐ, is
1) hydroxy,
2) C₁-C₁₀ alkoxy,
3) halogen,
4) nitro,
5) amino,
6) CF₃, or
7) carboxy,
and each cycloalkyl group is optionally substituted with a substituent independently selected from R_{b}, where R_{b} is
1) a group selected from Rₐ,
2) C₁-C₇ alkyl,
3) C₂-C₇ alkenyl,
4) C₂-C₇ alkynyl or
5) cyclic C₁-C₁₀ alkyl,
and each aryl is optionally substituted with R₁, to thus treat the urge incontinence in the subject.

2. The method of claim 1, wherein R₁ is methyl or ethyl;
wherein R₂ is H or fused benzene;
wherein R₃ is H, methyl, ethyl, propyl, tert-butyl, octyl, cyclohexyl, phenyl, hydroxy, methoxy, butoxy, pentoxy, phenoxy, benzoxy, trifluoromethyl ether, methylbenzene ether, 5-phenoxypentyloxy, 4-Hydroxypentyl, Cl, Br, F, or wherein R₂ and R₃ and the carbons to which they are attached form a fused benzene, fused 5,6-cyclohexenyl, fused cyclopentyl, or fused 2,3-furyl; and
wherein R₄ is H, methyl, ethyl, isopropyl, tert-butyl, 1-hydroxyethyl, ethoxy, butoxy, isopropoxy, phenoxy, benzyloxy, trifluoromethyl ether, Br, F, or wherein R₃ and R₄ and the carbons to which they are attached form a fused benzene, fused 5,6-cyclohexenyl, fused cyclopentyl, or fused 2,3-furyl.

3. The method of claim 1, wherein R₁ is methyl or ethyl;
wherein R₂ is H;
wherein R₃ is propyl, octyl, cyclohexyl, phenyl, hydroxy, methoxy, butoxy, pentoxy, phenoxy, benzoxy, trifluoromethyl ether, methylbenzene ether, 4-Hydroxypentyl, Cl, Br, F, or wherein R₂ and R₃ and the carbons to which they are attached form fused 5,6-cyclohexenyl, fused cyclopentyl, or fused 2,3-furyl; and
wherein R₄ is H, methyl, ethyl, isopropyl, tert-butyl, 1-hydroxy ethyl, ethoxy, butoxy, isopropoxy, phenyl, Br, F, or wherein R₃ and R₄ and the carbons to which they are attached form a fused 5,6-cyclohexenyl, fused cyclopentyl, or fused 2,3-furyl.

4. The method of claim 1, wherein R₁ is methyl or ethyl;
wherein R₂ is H;
wherein R₃ is cyclohexyl, benzoxy, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl; and
wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

5. The method of claim 2, wherein R₁ is methyl or ethyl;
wherein R₂ is H;
wherein R₃ is cyclohexyl, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl;
wherein R₄ is H, 1-hydroxyethyl, trifluoromethyl ether, or wherein R₃ and R₄ and the carbons to which they are attached form fused 5,6-cyclohexenyl, fused cyclopentyl, or fused 2,3-furyl.

6. The method of claim 1, wherein the compound has the structure: 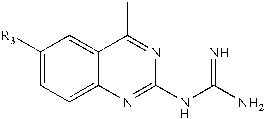
wherein R₃ is H, straight chained or branched C₁-C₇ alkyl or aryl.

7. The method of claim 6, wherein R₃ is butyl, sec-butyl, pentyl, hexyl, heptyl, or benzyl.

8. The method of claim 7, wherein R₃ is butyl, sec-butyl, hexyl, heptyl, or benzyl.

9. The method of claim 1, wherein the compound has the structure: 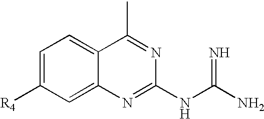
wherein R₄ is H, straight chained or branched C₁-C₇ alkyl.

10. The method of claim 10, wherein R₄ is H, or methyl.

11. The method of claim 1, wherein the compound has the structure: 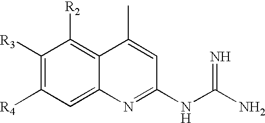
wherein R₂ is H or methyl;
wherein R₃ is H, straight chained or branched C₁-C₇ alkyl, aryl, alkoxy or halogen, or wherein R₂ and R₃ and the carbons to which they are attached form a fused aryl; and
wherein R₄ is H, methyl or halogen.

12. The method of claim 11, wherein R₂ is H, methyl;
wherein R₃ is H, Cl, methyl, ethyl, methoxy, phenyl or wherein R₂ and R₃ and the carbons to which they are attached form fused benzene; and
wherein R₄ is H, methyl or F.

13. The method of claim 1, wherein the compound has the structure: 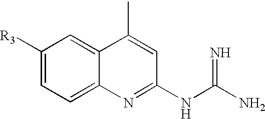
wherein R₃ is H, straight chained or branched C₁-C₇ alkyl.

14. The method of claim 14, wherein R₃ is butyl, pentyl or hexyl.

15. The method of claim 1, wherein the compound has the structure: 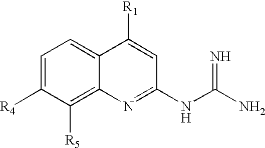
wherein R₁ is H, straight chained or branched C₁-C₇ alkyl; and
wherein each R₄ and R₅ is independently H or straight chained or branched C₁-C₇ alkyl.

16. The method of claim 16, wherein R₁ is methyl or ethyl; and
wherein each R₄ and R₅ is independently H or methyl.

17. The method of claim 1, wherein the compound has the structure:

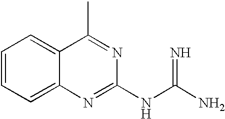

18. The method of claim 1, wherein the compound has the structure:

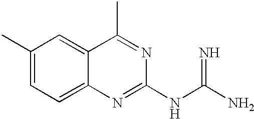

19. The method of claim 1, wherein the compound has the structure:

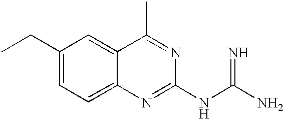

20. The method of claim 1, wherein the compound has the structure:

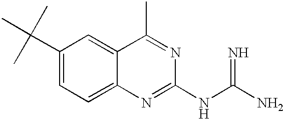

21. The method of claim 1, wherein the compound has the structure:

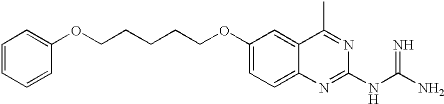

22. The method of claim 1, wherein the compound has the structure:

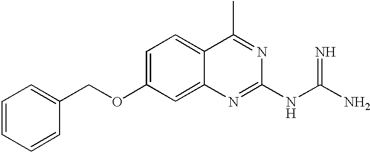

23. The method of claim 1, wherein the compound has the structure:

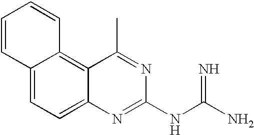

24. The method of claim 1, wherein the compound has the structure:

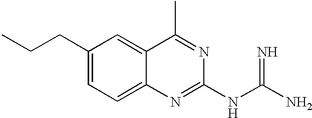

25. The method of claim 1, wherein the compound has the structure:

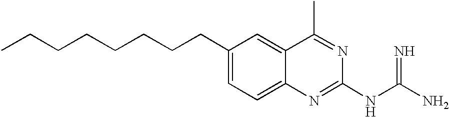

26. The method of claim 1, wherein the compound has the structure:

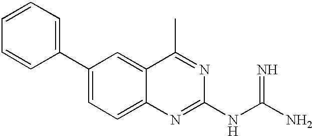

27. The method of claim 1, wherein the compound has the structure:

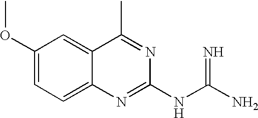

28. The method of claim 1, wherein the compound has the structure:

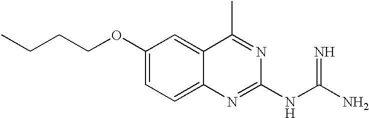

29. The method of claim 1, wherein the compound has the structure:

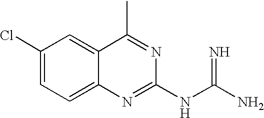

30. The method of claim 1, wherein the compound has the structure:

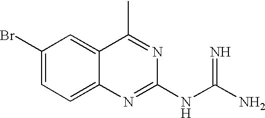

31. The method of claim 1, wherein the compound has the structure:

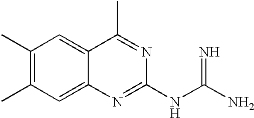

32. The method of claim 1, wherein the compound has the structure:

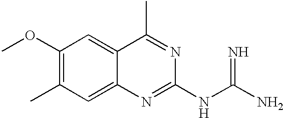

33. The method of claim 1, wherein the compound has the structure:

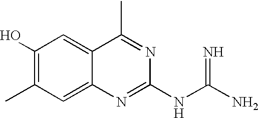

34. The method of claim 1, wherein the compound has the structure:

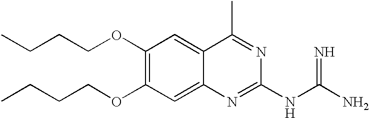

35. The method of claim 1, wherein the compound has the structure:

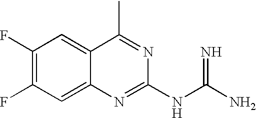

36. The method of claim 1, wherein the compound has the structure:

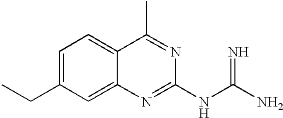

37. The method of claim 1, wherein the compound has the structure:

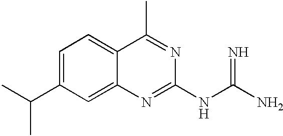

38. The method of claim 1, wherein the compound has the structure:

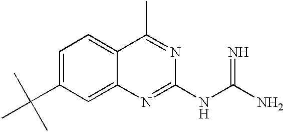

39. The method of claim 1, wherein the compound has the structure:

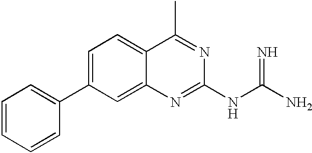

40. The method of claim 1, wherein the compound has the structure:

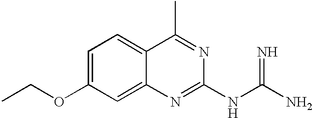

41. The method of claim 1, wherein the compound has the structure:

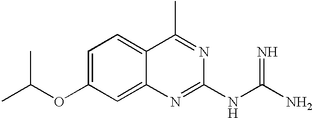

42. The method of claim 1, wherein the compound has the structure:

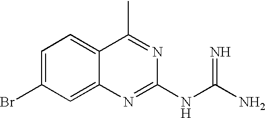

43. The method of claim 1, wherein the compound has the structure:

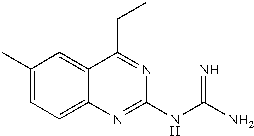

44. The method of claim 1, wherein the compound has the structure:

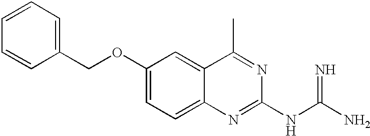

45. The method of claim 1, wherein the compound has the structure:

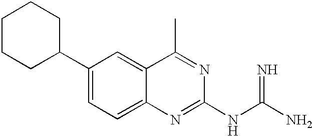

46. The method of claim 1, wherein the compound has the structure:

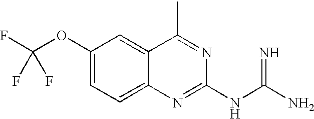

47. The method of claim 1, wherein the compound has the structure:

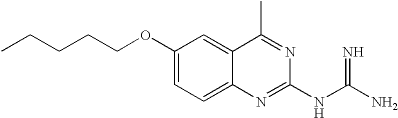

48. The method of claim 1, wherein the compound has the structure:

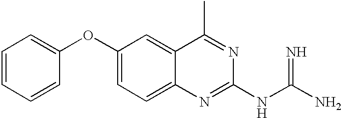

49. The method of claim 1, wherein the compound has the structure:

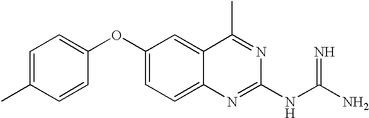

50. The method of claim 1, wherein the compound has the structure:

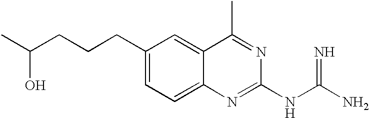

51. The method of claim 1, wherein the compound has the structure:

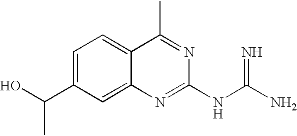

52. The method of claim 1, wherein the compound has the structure:

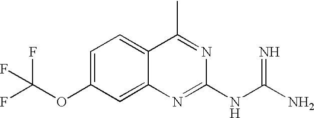

53. The method of claim 1, wherein the compound has the structure:

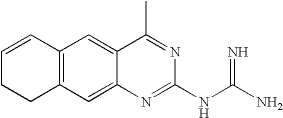

54. The method of claim 1, wherein the compound has the structure:

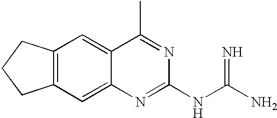

55. The method of claim 1, wherein the compound has the structure:

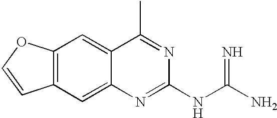

56. The method of claim 6, wherein the compound has the structure:

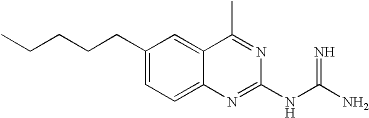

57. The method of claim 6, wherein the compound has the structure

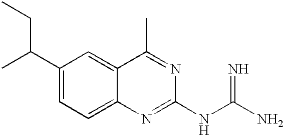

58. The method of claim 6, wherein the compound has the structure:

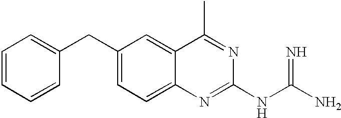

59. The method of claim 6, wherein the compound has the structure:

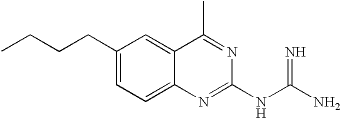

60. The method of claim 6, wherein the compound has the structure:

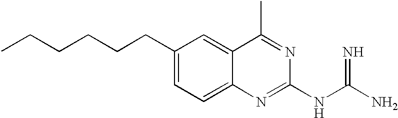

61. The method of claim 6, wherein the compound has the structure:

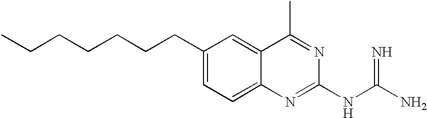

62. The method of claim 9, wherein the compound has the structure:

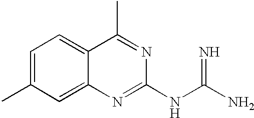

63. The method of claim 11, wherein the compound has the structure:

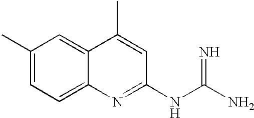

64. The method of claim 11, wherein the compound has the structure:

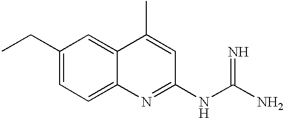

65. The method of claim 11, wherein the compound has the structure:

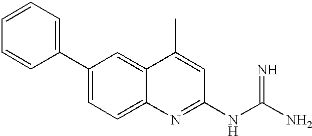

66. The method of claim 11, wherein the compound has the structure:

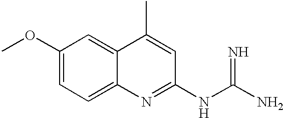

67. The method of claim 11, wherein the compound has the structure:

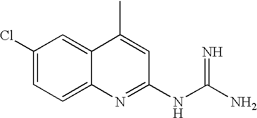

68. The method of claim 11, wherein the compound has the structure:

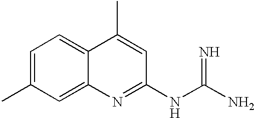

69. The method of claim 11, wherein the compound has the structure:

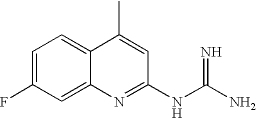

70. The method of claim 11, wherein the compound has the structure:

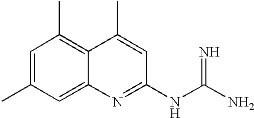

71. The method of claim 11, wherein the compound has the structure:

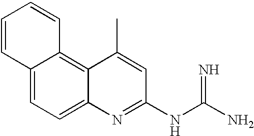

72. The method of claim 13, wherein the compound has the structure:

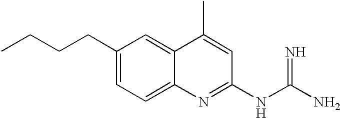

73. The method of claim 13, wherein the compound has the structure:

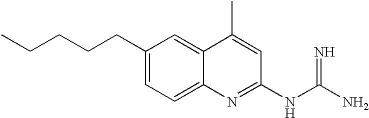

74. The method of claim 13, wherein the compound has the structure:

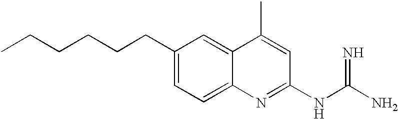

75. The method of claim 15, wherein the compound has the structure:

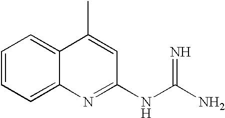

76. The method of claim 15, wherein the compound has the structure:

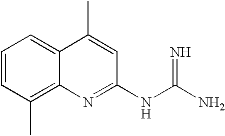

77. The method of claim 15, wherein the compound has the structure:

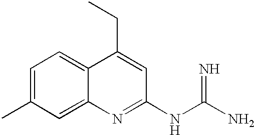

78. A method of treating pain in a subject in need of such treatment comprising administering to the subject an effective amount of a compound having the structure: 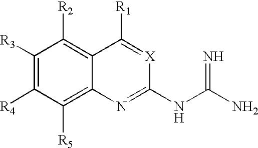
wherein X=CH, C(CH₃) or N;
wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;
and wherein each alkyl, alkenyl, alkynyl and alkoxy group is optionally substituted with a substituent independently selected from Rₐ, where Rₐ is
1) hydroxy,
2) C₁-C₁₀ alkoxy,
3) halogen,
4) nitro,
5) amino,
6) CF₃, or
7) carboxy,
and each cycloalkyl group is optionally substituted with a substituent independently selected from R_{b}, where R_{b} is
1) a group selected from Rₐ,
2) C₁-C₇ alkyl,
3) C₂-C₇ alkenyl,
4) C₂-C₇ alkynyl or
5) cyclic C₁-C₁₀ alkyl,
and each aryl is optionally substituted with R₁,
to thus treat pain in the subject.

79. A compound having the structure: 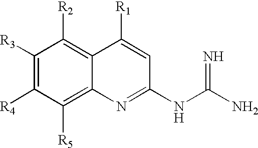
wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;
and wherein each alkyl, alkenyl, alkynyl and alkoxy group is optionally substituted with a substituent independently selected from Rₐ, where Rₐ is
1) hydroxy,
2) C₁-C₁₀ alkoxy,
3) halogen,
4) nitro,
5) amino,
6) CF₃, or
7) carboxy,
and each cycloalkyl group is optionally substituted with a substituent independently selected from R_{b}, where R_{b} is
1) a group selected from Rₐ,
2) C₁-C₇ alkyl,
3) C₂-C₇ alkenyl,
4) C₂-C₇ alkynyl or
5) cyclic C₁-C₁₀ alkyl,
and each aryl is optionally substituted with R₁.

80. The compound of claim 79, having the structure: 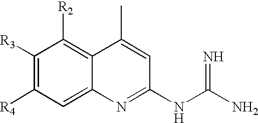
wherein R₂ is H or methyl;
wherein R₃ is H, straight chained or branched C₁-C₇ alkyl, aryl, alkoxy or halogen, or wherein R₂ and R₃ and the carbons to which they are attached form a fused aryl; and
wherein R₄ is H, methyl or halogen.

81. The compound of claim 79, wherein R₂ is H, methyl;
wherein R₃ is H, Cl, methyl, ethyl, methoxy, phenyl or wherein R₂ and R₃ and the carbons to which they are attached form fused benzene; and
wherein R₄ is H, methyl or F.

82. The compound of claim 79 having the structure: 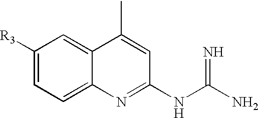
wherein R₃ is H, straight chained or branched C₁-C₇ alkyl.

83. The compound of claim 82, wherein R₃ is propyl, pentyl or hexyl.

84. The compound of claim 79 having the structure: 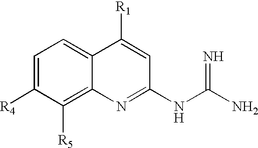
wherein R₁ is H, straight chained or branched C₁-C₇ alkyl; and
wherein each R₄ and R₅ is independently H or straight chained or branched C₁-C₇ alkyl.

85. The compound of claim 84, wherein R₁ is methyl or ethyl; and
wherein each R₄ and R₅ is independently H or methyl.

86. A compound having the structure: 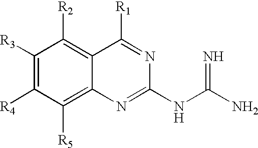
wherein each of R₁, R₂, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₃ is straight chained C₃, C₄, C₆ or C₇ alkyl or branched C₅-C₇ alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₂ and R₃ and the carbons to which they are attached form a fused cyclic alkyl or heterocyclic alkyl ring; or wherein R₃ and R₄ and the carbons to which they are attached form a fused aryl, heteroaryl, cyclic alkyl or heterocyclic alkyl ring; and
and wherein each alkyl, alkenyl, alkynyl and alkoxy group is optionally substituted with a substituent independently selected from Rₐ, where Rₐ is
1) hydroxy,
2) C₁-C₁₀ alkoxy,
3) halogen,
4) nitro,
5) amino,
6) CF₃, or
7) carboxy,
and each cycloalkyl group is optionally substituted with a substituent independently selected from R_{b}, where R_{b} is
1) a group selected from Rₐ,
2) C₁-C₇ alkyl,
3) C₂-C₇ alkenyl,
4) C₂-C₇ alkynyl or
5) cyclic C₁-C₁₀ alkyl,
and each aryl is optionally substituted with R₁.

87. The compound of claim 86 having the structure: 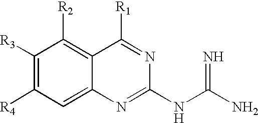
wherein R₁ is H, straight chained or branched C₁-C₇ alkyl;
wherein R₂ is H, straight chained or branched C₁-C₇ alkyl or fused aryl;
wherein R₃ is straight chained C₃, C₄, C₆ or C₇ alkyl or branched C₅-C₇ alkyl, cycloalkyl, substituted or unsubstituted aryl, hydroxyl, straight chained or branched alkoxy, halogenated ether, or halogen;
wherein R₄ is H, branched C₁-C₇ alkyl, aryl, straight chained or branched alkoxy or halogen; or wherein R₂ and R₃ and the carbons to which they are attached form a fused C₃-C₆ cyclic alkyl or heterocyclic alkyl ring; or wherein R₃ and R₄ and the carbons to which they are attached form a fused C₆-C₇ aryl or heteroaryl ring, a fused C₃-C₆ cyclic alkyl or heterocyclic alkyl ring.

88. The compound of claim 86, wherein R₁ is methyl or ethyl;
wherein R₂ is H or fused benzene;
wherein R₃ is cyclohexyl, phenyl, hydroxy, methoxy, butoxy, pentoxy, phenoxy, benzoxy, trifluoromethyl ether, methylbenzene ether, 4-Hydroxypentyl, Cl, Br, F, or wherein R₂ and R₃ and the carbons to which they are attached form fused 5,6-cyclohexenyl, fused cyclopentyl, or fused 2,3-furyl; and
wherein R₄ is H, isopropyl, tert-butyl, 1-hydroxyethyl, ethoxy, butoxy, isopropoxy, phenyl, Br, F, or wherein R₃ and R₄ and the carbons to which they are attached form fused 5,6-cyclohexenyl, fused cyclopentyl, or fused 2,3-furyl.

89. The compound of claim 86, wherein R₁ is methyl or ethyl;
wherein R₂ is H or fused benzene;
wherein R₃ is cyclohexyl, benzoxy, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl; and
wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

90. The compound of claim 86, wherein R₁ is methyl or ethyl;
wherein R₂ is H or fused benzene;
wherein R₃ is cyclohexyl, pentoxy, phenoxy, trifluoromethyl ether, methylbenzene ether, 4-hydroxypentyl, or wherein R₂ and R₃ and the carbons to which they are attached form fused 5,6-cyclohexenyl, fused cyclopentyl, or fused 2,3-furyl;
wherein R₄ is H, 1-hydroxyethyl, trifluoromethyl ether, or wherein R₃ and R₄ and the carbons to which they are attached form fused 5,6-cyclohexenyl, fused cyclopentyl or fused 2,3-furyl.

91. The compound of claim 86 having the structure: 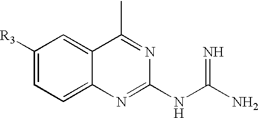
wherein R₃ is straight chained C₃, C₄, C₆ or C₇ alkyl or branched C₅-C₇ alkyl or aryl.

92. The compound of claim 91, wherein R₃ is butyl, hexyl, heptyl, or benzyl.

93. The compound of claim 86, having the structure:

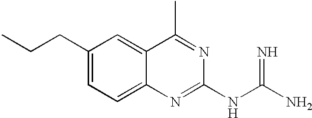

94. The compound of claim 86, having the structure:

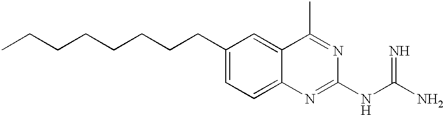

95. The compound of claim 86, having the structure:

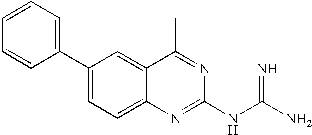

96. The compound of claim 86, having the structure:

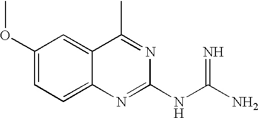

97. The compound of claim 86, having the structure:

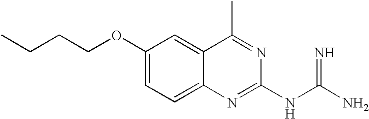

98. The compound of claim 86, having the structure:

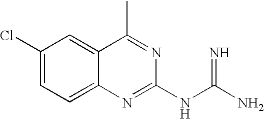

99. The compound of claim 86, having the structure:

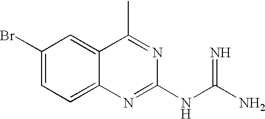

100. The compound of claim 86, having the structure:

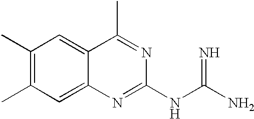

101. The compound of claim 86, having the structure:

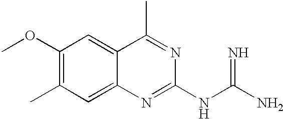

102. The compound of claim 86, having the structure:

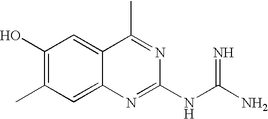

103. The compound of claim 86, having the structure:

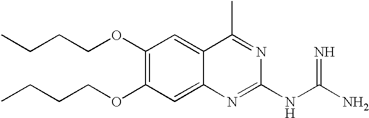

104. The compound of claim 86, having the structure:

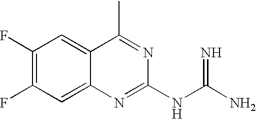

105. The compound of claim 86, having the structure:

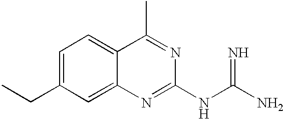

106. The compound of claim 86, having the structure:

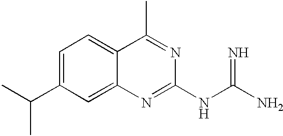

107. The compound of claim 86, having the structure:

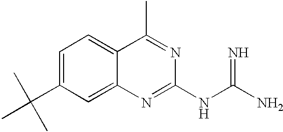

108. The compound of claim 86, having the structure:

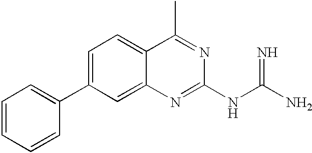

109. The compound of claim 86, having the structure:

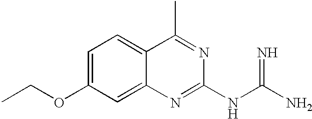

110. The compound of claim 86, having the structure:

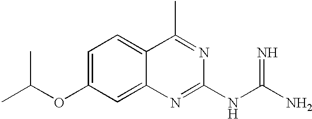

111. The compound of claim 86, having the structure:

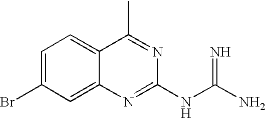

112. The compound of claim 86, having the structure:

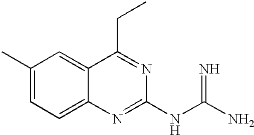

113. The compound of claim 86, having the structure:

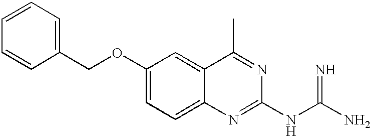

114. The compound of claim 86, having the structure:

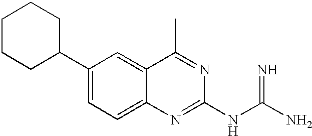

115. The compound of claim 86, having the structure:

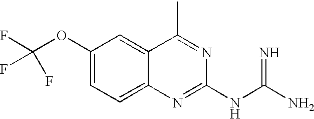

116. The compound of claim 86, having the structure:

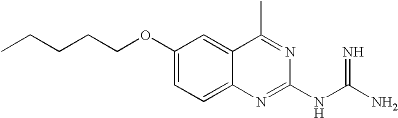

117. The compound of claim 86, having the structure:

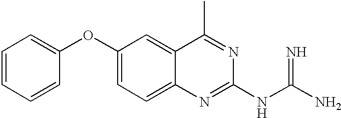

118. The compound of claim 86, having the structure:

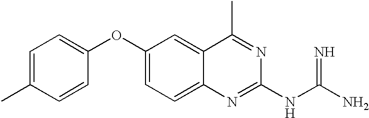

119. The compound of claim 86, having the structure:

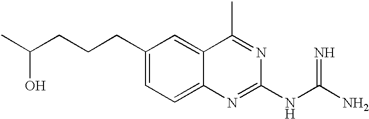

120. The compound of claim 86, having the structure:

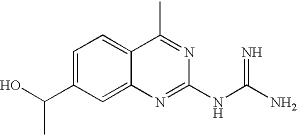

121. The compound of claim 86, having the structure:

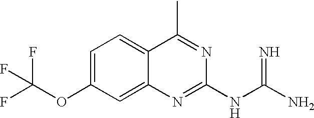

122. The compound of claim 86, having the structure:

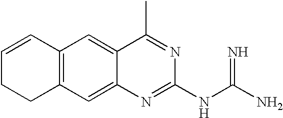

123. The compound of claim 86, having the structure:

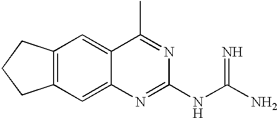

124. The compound of claim 86, having the structure:

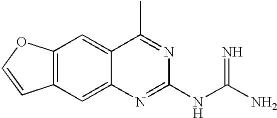

125. The compound of claim 91, having the structure:

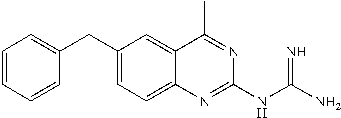

126. The compound of claim 91, having the structure:

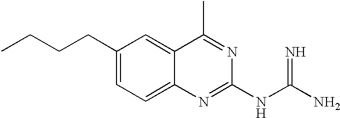

127. The compound of claim 91, having the structure:

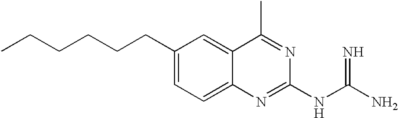

128. The compound of claim 91, having the structure:

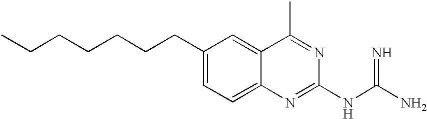

129. The compound of claim 79, having the structure:

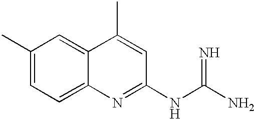

130. The compound of claim 79, having the structure:

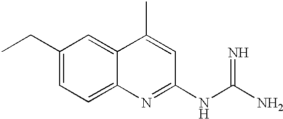

131. The compound of claim 79, having the structure:

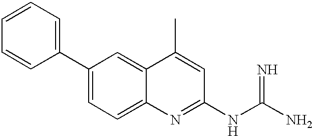

132. The compound of claim 79, having the structure:

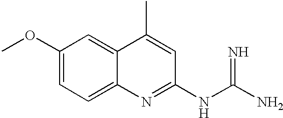

133. The compound of claim 79, having the structure:

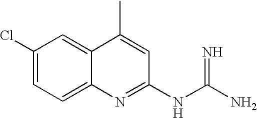

134. The compound of claim 79, having the structure:

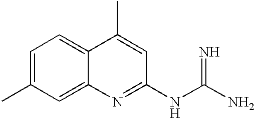

135. The compound of claim 79, having the structure:

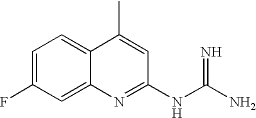

136. The compound of claim 79, having the structure:

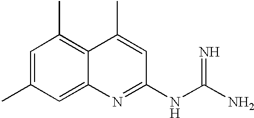

137. The compound of claim 79, having the structure:

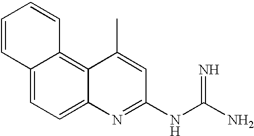

138. The compound of claim 79, having the structure:

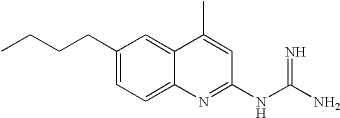

139. The compound of claim 79, having the structure:

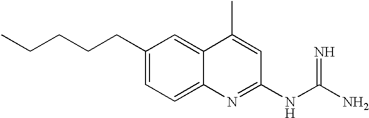

140. The compound of claim 79, having the structure:

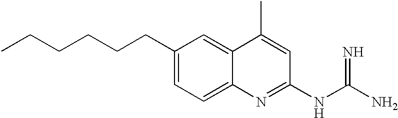

141. The compound of claim 79, having the structure:

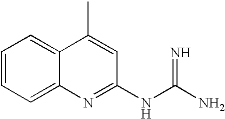

142. The compound of claim 79, having the structure:

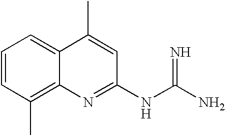

143. The compound of claim 79, having the structure:

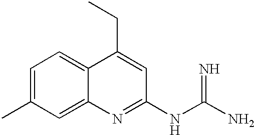

144. A compound having the structure: 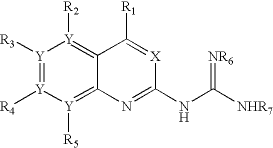
wherein X=CH, C(CH₃) or N;
wherein each of R₁, R₂, R₃, R₄ and R₅ is independently H, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl, hydroxy, halogenated ether, nitro, amino, halogen, —CN, —C(═Z)R₆, —C(═Z)OR₆, —C(═Z)N(R₆)₂, —N(R₆)—C(═Z)R₆, —N(R₆)—C(═Z)N(R₆)₂, —OC(═Z)R₆, —C(═Z)OR₆—OR₆ or —SR₆;
wherein Z is O or S; and
wherein R₆ is C₁-C₁₀ straight chained or branched alkyl, aryl, (CH₂)ₙQ, C₂-C₁₀ alkenyl, C₃-C₁₀ cycloalkyl, C₅-C₁₀ cycloalkenyl,
wherein Q is OR₇, SR₇, N(R₇)₂ or aryl,
wherein R₇ is H, alkyl, alkenyl, alkynyl, cycloalkyl, cycloalkenyl, aryl,
wherein R₂ and R₃ and the carbons to which they are attached form a fused aryl, heteroaryl, C₅-C₁₀ cyclic alkyl or heterocyclic alkyl ring; or wherein R₃ and R₄ and the carbons to which they are attached form a fused aryl, heteroaryl, cyclic alkyl or heterocyclic alkyl ring;
and wherein each alkyl, alkenyl, alkynyl and alkoxy group is optionally substituted with a substituent independently selected from Rₐ, where Rₐ is
1) hydroxy,
2) C₁-C₁₀ alkoxy,
3) halogen,
4) nitro,
5) amino,
6) CF₃, or
7) carboxy,
and each cycloalkyl group is optionally substituted with a substituent independently selected from R_{b}, where R_{b} is
1) a group selected from Rₐ,
2) C₁-C₇ alkyl,
3) C₂-C₇ alkenyl,
4) C₂-C₇ alkynyl or
5) cyclic C₁-C₁₀ alkyl,
and each aryl is optionally substituted with R₁, and
wherein each R₆ and R₇ is independently acetate, formate, phosphate ester, dimethylglycine ester, aminoalkylbenzyl ester, aminoalkyl ester and carboxyalkyl ester.

145. The compound of claim 144, wherein R₆ and R₇ is independently acetyl or acyl.

146. A pharmaceutical composition comprising the compound of any one of claims 78-143 and a pharmaceutically acceptable carrier.

147. The pharmaceutical composition of claim 146, wherein the carrier is phosphate buffered saline, physiological saline or water.

148. A method of preparing a pharmaceutical composition comprising mixing the compound of any one of claims 78-143 with a pharmaceutical acceptable carrier.

149. The method of claim 148, wherein the carrier is phosphate buffered saline, physiological saline or water.

150. A compound which is converted in vivo to the compound of any one of claims 78-143.

151. A compound which is a metabolite of the compound of any one of claims 78-143.

152. A salt of the compound of any one of claims 78-143.
